(19) United States
(12) Patent Application Publication
(10) Pub. No.: US 2026/0008868 A1
(43) Pub. Date: Jan. 8, 2026

(54) 5T4-BINDING PROTEINS

(71) Applicant: Solve Therapeutics, Inc., San Diego, CA (US)

(72) Inventors: Jeffry D. WATKINS, Encinitas, CA (US); Andrew POW, Encinitas, CA (US); Brian LANNUTTI, Solana Beach, CA (US); Katti JESSEN, San Diego, CA (US)

(73) Assignee: Solve Therapeutics, Inc., San Diego, CA (US)

(21) Appl. No.: 19/257,097

(22) Filed: Jul. 1, 2025

Related U.S. Application Data
(60) Provisional application No. 63/668,058, filed on Jul. 5, 2024.
(60) Provisional application No. 63/723,077, filed on Nov. 20, 2024.
(60) Provisional application No. 63/723,081, filed on Nov. 20, 2024.
(60) Provisional application No. 63/743,001, filed on Jan. 8, 2025.

(30) Foreign Application Priority Data
Jul. 5, 2024 (WO) PCT/US2024/036950

(51) Int. Cl. C07K 16/30; A61P 35/00

(52) U.S. Cl. CPC C07K 16/30; A61P 35/00; C07K 2317/24; C07K 2317/77; C07K 2317/92; C07K 2317/94

(57) ABSTRACT

The present disclosure provides recombinant proteins that bind to 5T4 and methods of using them to treat cancer.

### CROSS-REFERENCE TO RELATED APPLICATION

This application claims priority to U.S. Provisional Patent Application 63/668,058, filed Jul. 5, 2024; U.S. Provisional Patent Application 63/723,081, filed Nov. 20, 2024; U.S. Provisional Patent Application 63/723,077, filed Nov. 20, 2024; U.S. Provisional Patent Application 63/743,001, filed Jan. 8, 2025; and PCT Patent Application PCT/US2024/036950, filed Jul. 5, 2024. The disclosures of those priority applications are incorporated by reference herein in their entirety.

### SEQUENCE LISTING

The instant application contains a Sequence Listing which has been submitted electronically in .XML format, is part of the specification, and is hereby incorporated by reference herein in its entirety. Said .XML copy, created on Jun. 27, 2025, is named 122878.US020.xml and is 101,556 bytes in size.

### BACKGROUND OF THE INVENTION

5T4, also known as trophoblast glycoprotein (TBGP) or Wnt-Activated Inhibitory Factor 1 (WAIF1), is a glycoprotein that is a member of the leucine-rich repeat (LRR)-containing family of proteins. As an antagonist of the Wnt/β-catenin signaling pathway, 5T4 plays a role in cell adhesion, shape, and motility. 5T4 is expressed in numerous cancers including breast, ovarian, lung, colorectal, bladder, and gastric cancers and on placental trophoblast, but rarely in normal adult tissues. Given its expression pattern, 5T4 is an ideal candidate target for cancer therapy.

5T4 expression has been associated with poor prognosis and higher metastatic potential in patients with various cancers, and thus represents a promising therapeutic target. In view of the critical role of 5T4 in tumor progression, there is a need for new and improved cancer immunotherapies that target 5T4.

### SUMMARY OF THE INVENTION

The present disclosure provides recombinant proteins that bind specifically to 5T4. In some embodiments, the present 5T4-binding protein is an anti-5T4 antibody or an antigen-binding portion thereof.

In some embodiments, the present disclosure provides an isolated anti-5T4 antibody or an antigen-binding portion thereof, wherein said antibody binds to the same epitope as, or competes for binding to human 5T4 with, an antibody comprising a heavy chain variable domain (V_{H}) and a light chain variable domain (V_{L}) that comprise SEQ ID NOs:1 and 2, respectively.

In certain embodiments, the antibody comprises heavy chain CDR (HCDR) 1-3 and light chain CDR (LCDR) 1-3 comprising SEQ ID NOs:47, 52, 57, 60, 63, and 66, respectively.

In certain embodiments, the antibody comprises a V_{H} and a V_{L} comprising SEQ ID NOs:1 and 2, respectively.

In some embodiments, the present disclosure provides an isolated anti-5T4 antibody or an antigen-binding portion thereof, wherein said antibody binds to the same epitope as, or competes for binding to human 5T4 with, an antibody comprising a heavy chain variable domain (V_{H}) and a light chain variable domain (V_{L}) that comprise SEQ ID NOs:3 and 4, respectively.

In certain embodiments, the antibody comprises heavy chain CDR (HCDR) 1-3 and light chain CDR (LCDR) 1-3 comprising SEQ ID NOs:68, 74, 79, 82, 85, and 88, respectively.

In certain embodiments, the antibody comprises a V_{H} and a V_{L} comprising SEQ ID NOs:3 and 4, respectively.

In certain embodiments, the antibody comprises a V_{H} comprising SEQ ID NOs:73, 75, and 80, and a V_{L} comprising SEQ ID NOs:83, 86, and 88. The antibody or portion may be humanized. In particular embodiments, the V_{H} comprises framework regions derived from a human IGHV1-69*02 germline gene, and the V_{H} may comprise one or more back mutations selected from V20I, G44S, M48I, V67A, I69L, A71V, T75S, and R94S, wherein the numbering is according to Kabat. Additionally or alternatively, in particular embodiments, the V_{L} comprises framework regions derived from a human IGKV2-30*01 germline gene, and the V_{L} may comprise one or more back mutations selected from F36L and Y49S, wherein the numbering is according to Kabat.

In certain embodiments, an anti-5T4 antibody herein comprises,
a) a V_{H} that comprises any one of SEQ ID NOs:3, 29, 31, and 33, or an amino acid sequence at least 95% identical thereto;
b) a V_{L} that comprises any one of SEQ ID NOs:4, 30, 32, and 34, or an amino acid sequence at least 95% identical thereto; or
c) a) and b).
   For example, the V_{H} and the V_{L} may be at least 95% identical in sequence to
SEQ ID NOs:3 and 4, respectively,
SEQ ID NOs:29 and 30, respectively,
SEQ ID NOs:31 and 32, respectively, or
SEQ ID NOs:33 and 34, respectively.
   In particular embodiments, the V_{H} and the V_{L} may comprise
SEQ ID NOs:3 and 4, respectively,
SEQ ID NOs:29 and 30, respectively,
SEQ ID NOs:31 and 32, respectively, or
SEQ ID NOs:33 and 34, respectively.

In some embodiments, an anti-5T4 antibody herein comprises a human IgG₁ constant region. In certain embodiments, the human IgG₁ constant region comprises SEQ ID NO:101, optionally without the C-terminal lysine. In certain embodiments, the human IgG₁ constant region comprises SEQ ID NO:102, optionally without the C-terminal lysine.

In some embodiments, an anti-5T4 antibody herein comprises a human light chain constant region that comprises SEQ ID NO:103.

In particular embodiments, the present disclosure provides an isolated anti-5T4 antibody comprising a heavy chain that comprises the amino acid sequences of SEQ ID NOs:1 and 101, optionally without the C-terminal lysine, and a light chain that comprises the amino acid sequences of SEQ ID NOs:2 and 103.

In particular embodiments, the present disclosure provides an isolated anti-5T4 antibody comprising a heavy chain that comprises the amino acid sequences of SEQ ID NOs:3 and 101, optionally without the C-terminal lysine, and a light chain that comprises the amino acid sequences of SEQ ID NOs:4 and 103.

In particular embodiments, the present disclosure provides an isolated anti-5T4 antibody comprising a heavy chain that comprises the amino acid sequences of SEQ ID NOs:29 and 101, optionally without the C-terminal lysine, and a light chain that comprises the amino acid sequences of SEQ ID NOs:30 and 103.

In particular embodiments, the present disclosure provides an isolated anti-5T4 antibody comprising a heavy chain that comprises the amino acid sequences of SEQ ID NOs:31 and 101, optionally without the C-terminal lysine, and a light chain that comprises the amino acid sequences of SEQ ID NOs:32 and 103.

In particular embodiments, the present disclosure provides an isolated anti-5T4 antibody comprising a heavy chain that comprises the amino acid sequences of SEQ ID NOs:33 and 101, optionally without the C-terminal lysine, and a light chain that comprises the amino acid sequences of SEQ ID NOs:34 and 103.

In particular embodiments, the present disclosure provides an isolated anti-5T4 antibody comprising a heavy chain that comprises the amino acid sequences of SEQ ID NOs:1 and 102, optionally without the C-terminal lysine, and a light chain that comprises the amino acid sequences of SEQ ID NOs:2 and 103.

In particular embodiments, the present disclosure provides an isolated anti-5T4 antibody comprising a heavy chain that comprises the amino acid sequences of SEQ ID NOs:3 and 102, optionally without the C-terminal lysine, and a light chain that comprises the amino acid sequences of SEQ ID NOs:4 and 103.

In particular embodiments, the present disclosure provides an isolated anti-5T4 antibody comprising a heavy chain that comprises the amino acid sequences of SEQ ID NOs:29 and 102, optionally without the C-terminal lysine, and a light chain that comprises the amino acid sequences of SEQ ID NOs:30 and 103.

In particular embodiments, the present disclosure provides an isolated anti-5T4 antibody comprising a heavy chain that comprises the amino acid sequences of SEQ ID NOs:31 and 102, optionally without the C-terminal lysine, and a light chain that comprises the amino acid sequences of SEQ ID NOs:32 and 103.

In particular embodiments, the present disclosure provides an isolated anti-5T4 antibody comprising a heavy chain that comprises the amino acid sequences of SEQ ID NOs:33 and 102, optionally without the C-terminal lysine, and a light chain that comprises the amino acid sequences of SEQ ID NOs:34 and 103.

In some embodiments, an isolated antibody or portion described herein may be conjugated to a cytotoxin or a detectable label.

The present disclosure also provides a pharmaceutical composition comprising an isolated antibody or portion described herein and a pharmaceutically acceptable excipient.

Further, the present disclosure provides nucleic acid molecule(s) encoding an isolated antibody or portion described herein, expression vector(s) comprising the nucleic acid molecule(s), and a recombinant host cell comprising the expression vector(s). The present disclosure also provides a method of making an anti-5T4 antibody or an antigen-binding portion thereof, comprising:
culturing a recombinant host cell described herein under conditions that allow expression of the antibody or portion, and
isolating the antibody or portion from the cell culture.

The present disclosure also provides a method of treating a 5T4-positive cancer in a patient in need thereof (e.g., a human patient), comprising administering to the patient an anti-5T4 antibody or antigen-binding portion thereof described herein or a pharmaceutical composition described herein. Also provided are use of an anti-5T4 antibody or antigen-binding portion thereof described herein for the manufacture of a medicament for treating a 5T4-positive cancer in a patient in need thereof, or an anti-5T4 antibody or antigen-binding portion thereof described herein or a pharmaceutical composition described herein for use in treating a 5T4-positive cancer in a patient in need thereof. In certain embodiments, the 5T4-positive cancer is selected from adenocarcinoma, bladder cancer, bowel cancer, breast cancer (e.g., triple negative breast cancer), cervical cancer, colorectal cancer, esophageal cancer, gastric cancer, head and neck cancer (e.g., head and neck squamous cell carcinoma), lung cancer (e.g., non-small cell lung cancer), mesothelioma, ovarian cancer, pancreatic cancer, prostate cancer, renal cancer, squamous cell carcinoma, uterine cancer, retinoblastoma, and Ewing sarcoma.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a pair of tables showing CDRs defined according to different systems for antibody SLX-2079. SEQ: SEQ ID NO.

FIG. 2 is a pair of tables showing CDRs defined according to different systems for antibody SLX-2104. SEQ: SEQ ID NO. Grafted: sequence grafted to human acceptor frameworks during humanization.

FIG. 3 is a sequence alignment of the heavy and light chain variable domains of antibody SLX-2104 with the most homologous human heavy and light chain germline sequences (IGHV1-69*02 and IGKV2-30*01, respectively). CDRs grafted to human acceptor sequences for humanization are in boldface. Asterisks indicate potential positions for making back mutations during humanization.

FIG. 4 is a table showing humanized versions of SLX-2104 with various combinations of V_{H} and V_{L} variants having the indicated framework back mutations.

FIG. 5 is a line graph showing internalization of the listed antibodies on NCI-H226 cells as measured by the increase in non-quenched fluorescence signal over time (AMFI).

FIG. 6 is a line graph showing internalization of the listed antibodies on NCI-H520 cells as measured by the increase in non-quenched fluorescence signal over time (AMFI).

### DETAILED DESCRIPTION OF THE INVENTION

The present disclosure provides novel proteins that specifically bind to 5T4 (“5T4-binding proteins”). These proteins may be antibodies or comprise antigen-binding portions thereof. The proteins can be used to treat cancer (e.g., a 5T4-positive cancer) in patients in need thereof.

The term “antibody” (Ab) or “immunoglobulin” (Ig), as used herein, refers to a tetramer comprising two heavy (H) chains (about 50-70 kDa) and two light (L) chains (about 25 kDa) inter-connected by disulfide bonds. Each heavy chain is comprised of a heavy chain variable domain (V_{H}) and a heavy chain constant region (C_{H}). Each light chain is composed of a light chain variable domain (V_{L}) and a light chain constant region (C_{L}). The V_{H} and V_{L} domains can be subdivided further into regions of hypervariability, termed “complementarity-determining regions” (CDRs), interspersed with regions that are more conserved, termed “framework regions” (FRs). Each V_{H} and V_{L} is composed of three CDRs (HCDR herein designates a CDR from the heavy chain; and LCDR herein designates a CDR from the light chain) and four FRs, arranged from amino-terminus to carboxy-terminus in the following order: FR1, CDR1, FR2, CDR2, FR3, CDR3, FR4. The amino acid position numbers and FRs and CDRs in the heavy or light chain may be defined in accordance with the IMGT® system (Lefranc et al., Dev Comp Immunol. (2003) 27(1):55-77); the Kabat system (Sequences of Proteins of Immunological Interest, National Institutes of Health, Bethesda, MD (1987 and 1991)); the Chothia system (Chothia & Lesk, J Mol Biol. (1987) 196:901-17; Chothia et al., Nature (1989) 342:878-83); Abhinandan et al., Molecular Immunology (2008) 45(14):3832-39; MacCallum et al., J Mol Biol. (1996) 262:732-45; or Honegger and Phickthun, J Mol Biol. (2001) 309(3):657-70. The CDR boundaries of various schemes are illustrated in Table 1, where the amino acid numbers are Kabat numbers unless otherwise indicated.

TABLE 1
CDR Delineations According to Various Schemes

| CDR | Kabat | AbM | Chothia | Contact |
|---|---|---|---|---|
| LCDR1 | L24-L34 | L24-L34 | L26-L32 | L30-L36 |
| LCDR2 | L50-L56 | L50-L56 | L50-L52 | L46-L55 |
| LCDR3 | L89-L97 | L89-L97 | L91-L96 | L89-L96 |
| HCDR1 (Kabat | H31-H35B | H26-H35B | H26-H32 | H30-H35B |
| nos.) | | | | |
| HCDR1 (Chothia | H31-H35 | H26-H35 | H26-H32 | H30-H35 |
| nos.) | | | | |
| HCDR2 | H50-H65 | H50-H58 | H53-H55 | H47-H58 |
| HCDR3 | H95-H102 | H95-H102 | H95-H101 | H93-H101 |

The term “recombinant antibody” or “recombinant protein” refers to a non-naturally occurring antibody or protein that is expressed from a cell or cell line comprising one or more nucleotide sequences that encode the antibody or protein, wherein the cell or cell line does not naturally comprise the nucleotide sequence(s).

The term “isolated protein,” “isolated polypeptide” or “isolated antibody” refers to a protein, polypeptide or antibody that by virtue of its origin or source of derivation (1) is not associated with components that naturally accompany it in its native state, (2) is free of other proteins from the same species, (3) is expressed by a cell from a different species, and/or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “affinity” refers to a measure of the attraction between two molecules, e.g., between an antigen and an antibody. The intrinsic attractiveness of an antibody for an antigen is typically expressed as the binding affinity equilibrium constant (K_{D}) of the antibody-antigen interaction. An antibody is said to specifically bind to an antigen when the K_{D} for the binding is ≤1 μM, e.g., ≤100 nM or ≤10 nM. A K_{D} binding affinity constant can be measured, e.g., by surface plasmon resonance (SPR) using, for example, the Biacore™ system, the IBIS-MX96 SPR system from IBIS Technologies or the Carterra LSA SPR platform, or by Bio-Layer Interferometry (BLI) using, for example, the Octet™ system from ForteBio.

The term “epitope” as used herein refers to a portion (determinant) of an antigen that specifically binds to an antibody or a related molecule such as a bi-specific binding molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between a protein (e.g., an antigen) and an interacting molecule (e.g., an antibody) occur linearly along the primary amino acid sequence of the protein. In a conformational epitope, the points of interaction occur across amino acid residues on the protein that are separated from one another in the primary amino acid sequence. Once a desired epitope on an antigen is determined, it is possible to generate antibodies to that epitope using techniques well known in the art. For example, an antibody to a linear epitope may be generated, e.g., by immunizing an animal with a peptide having the amino acid residues of the linear epitope. An antibody to a conformational epitope may be generated, e.g., by immunizing an animal with a mini-domain containing the relevant amino acid residues of the conformational epitope. An antibody to a particular epitope can also be generated, e.g., by immunizing an animal with the target molecule of interest (e.g., 5T4) or a relevant portion thereof, then screening for binding to the epitope. An antibody to a particular epitope also may be generated using phage display methods.

One can determine whether an antibody binds to the same epitope as or competes for binding with an anti-5T4 antibody of the invention by using methods known in the art, including, without limitation, competition assays, epitope binning, and alanine scanning. In one embodiment, one allows the anti-5T4 antibody of the invention to bind to 5T4 under saturating conditions, and then measures the ability of the test antibody to bind to 5T4. If the test antibody is able to bind to 5T4 at the same time as the reference anti-5T4 antibody, then the test antibody binds to a different epitope than the reference anti-5T4 antibody. However, if the test antibody is not able to bind to 5T4 at the same time, then the test antibody may bind to the same epitope, an overlapping epitope, or an epitope that is in close proximity to the epitope bound by the anti-5T4 antibody of the invention. This experiment can be performed using, e.g., ELISA, RIA, Biacore™, SPR, BLI, or flow cytometry. To test whether an anti-5T4 antibody cross-competes with another anti-5T4 antibody, one may use the competition method described above in two directions, i.e., determining if the known antibody blocks the test antibody and vice versa. Such cross-competition experiments may be performed, e.g., using an IBIS MX96 or Carterra LSA SPR instrument or the Octet™ system.

The term “antigen-binding portion” of an antibody (or simply “antibody portion”), as used herein, refers to one or more portions or fragments of an antibody that retain the ability to specifically bind to the antigen (e.g., human 5T4, or a portion thereof) of the antibody. It has been shown that certain fragments of a full-length antibody can perform the antigen-binding function of the antibody. Examples of binding fragments encompassed within the term “antigen-binding portion” include (i) a Fab fragment: a monovalent fragment consisting of the V_{L}, V_{H}, C_{L} and C_{H1} domains; (ii) a F(ab′)2 fragment: a bivalent fragment comprising two Fab fragments linked by a disulfide bridge at the hinge region; (iii) an Fd fragment consisting of the V_{H} and C_{H1} domains; (iv) an Fv fragment consisting of the V_{L} and V_{H} domains of a single arm of an antibody, (v) a dAb fragment, which consists of a V_{H} domain; and (vi) an isolated CDR capable of specifically binding to an antigen. Furthermore, although the two domains of the Fv fragment, V_{L} and V_{H}, are encoded by separate genes, they can be joined, using recombinant methods, by a synthetic linker that enables them to be made as a single polypeptide chain in which the V_{L} and V_{H} domains pair to form monovalent molecules (known as single chain Fv (scFv)). Also within the present disclosure are antigen-binding molecules comprising a V_{H} and/or a V_{L}. In the case of a V_{H}, the molecule may also comprise one or more of a C_{H1}, hinge, C_{H2}, or C_{H3} region. Such single chain antibodies are also intended to be encompassed within the term “antigen-binding portion” of an antibody. Other forms of single chain antibodies, such as diabodies, are also encompassed. Diabodies are bivalent, bi-specific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen-binding sites.

Antibody portions, such as Fab and F(ab′)₂ fragments, can be prepared from whole antibodies using conventional techniques, such as papain or pepsin digestion of whole antibodies. Moreover, antibodies, antibody portions and immunoadhesin molecules can be obtained using standard recombinant DNA techniques, e.g., as described herein.

In some embodiments, antibodies may originate from non-human sources and may have been humanized to reduce anti-antibody immune responses. The term “humanize” refers to the fact that where an antibody is wholly or partially of non-human origin (for example, a murine or chicken antibody obtained from immunization of mice or chickens, respectively, with an antigen of interest, or a chimeric antibody based on such a murine or chicken antibody), it is possible to replace certain amino acids, in particular in the framework regions and constant regions of the heavy and light chains, in order to avoid or minimize an immune response in humans. Although it is not possible to precisely predict the immunogenicity, and thereby the human anti-antibody response, of a particular antibody, non-human antibodies tend to be more immunogenic in humans than human antibodies. Antibodies of non-human origin thus can be humanized to reduce the risk of a human anti-antibody response.

Humanization typically involves modification of the framework regions of the variable domain sequences. Amino acid residues that are part of complementarity determining regions (CDRs) most often will not be altered in connection with humanization, although in certain cases it may be desirable to alter individual CDR amino acid residues, for example to remove a glycosylation site, a deamidation site, an aspartate isomerization site or an undesired cysteine or methionine residue. N-linked glycosylation occurs by attachment of an oligosaccharide chain to an asparagine residue in the tripeptide sequence Asn-X-Ser or Asn-X-Thr, where X may be any amino acid except Pro. Removal of an N-glycosylation site may be achieved by mutating either the Asn or the Ser/Thr residue to a different residue, preferably by way of conservative substitution. Deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ser, Asn-Thr, and Asn-Ala. When such a deamidation site, in particular Asn-Gly, is present in a CDR sequence, it may therefore be desirable to remove the site, typically by conservative substitution to remove one of the implicated residues.

The class (isotype) and subclass of anti-5T4 antibodies may be determined by any method known in the art. In general, the class and subclass of an antibody may be determined using antibodies that are specific for a particular class and subclass of antibody. Such antibodies are available commercially. The class and subclass can be determined by ELISA or Western blot as well as other techniques. Alternatively, the class and subclass may be determined by sequencing all or a portion of the constant regions of the heavy and/or light chains of the antibodies, comparing their amino acid sequences to the known amino acid sequences of various classes and subclasses of immunoglobulins, and determining the class and subclass of the antibodies.

The term “monoclonal antibody” or “monoclonal antibody composition” means a preparation of antibody molecules of single molecular composition, which displays a single binding specificity and affinity for a particular epitope.

Unless otherwise indicated, all antibody variable region amino acid residue numbers referred to in this disclosure are those under the Kabat numbering scheme. Eu numbering is employed for constant regions.

### L. 5T4-Binding Proteins

The present disclosure provides 5T4-binding proteins (e.g., anti-5T4 antibodies and proteins comprising the antibodies or antigen-binding portions thereof). Unless otherwise stated, “5T4” refers to human 5T4 herein. A human 5T4 polypeptide sequence is available under UniProt Accession No. Q13641 (TPBG_HUMAN) and is shown below:

(SEQ ID NO: 104)
MPGGCSRGPA AGDGRLRLAR LALVLLGWVS SSSPTSSASS
FSSSAPFLAS AVSAQPPLPD QCPALCECSE AARTVKCVNR
NLTEVPTDLP AYVRNLFLTG NQLAVLPAGA FARRPPLAEL
AALNLSGSRL DEVRAGAFEH LPSLRQLDLS HNPLADLSPF
AFSGSNASVS APSPLVELIL NHIVPPEDER QNRSFEGMVV
AALLAGRALQ GLRRLELASN HFLYLPRDVL AQLPSLRHLD
LSNNSLVSLT YVSFRNLTHL ESLHLEDNAL KVLHNGTLAE
LQGLPHIRVF LDNNPWVCDC HMADMVTWLK ETEVVQGKDR
LTCAYPEKMR NRVLLELNSA DLDCDPILPP SLQTSYVELG
IVLALIGAIF LLVLYLNRKG IKKWMHNIRD ACRDHMEGYH
YRYEINADPR LTNLSSNSDV

In the above sequence, amino acids 1-30 correspond to the signal sequence; amino acids 31-355 correspond to the extracellular domain (ECD); amino acids 356-376 correspond to the transmembrane domain; and amino acids 377-420 correspond to the intracellular domain. The 5T4-binding proteins herein bind to the ECD of 5T4.

The present disclosure provides antibodies directed against 5T4, and antigen-binding portions thereof. One advantage of novel anti-5T4 antibodies of the invention is that they may effectively bind to tumor cells; see, e.g., Examples 2, 5, and 7. Furthermore, the novel anti-5T4 antibodies of the invention may be effectively internalized in tumor cell lines as measured by the increase in non-quenched fluorescent antibody (non-surface exposed antibody) over time; see, e.g., Example 8.

In some embodiments, the present disclosure provides an anti-5T4 antibody or an antigen-binding portion thereof that competes or cross-competes for binding to human 5T4 with, or binds to the same epitope of human 5T4 as, any one of SLX-2079, SLX-2104, SLX-2024, SLX-2047, SLX-2052, SLX-2065, SLX-2077, SLX-2089, SLX-2095, SLX-2105, SLX-2113, SLX-2121, SLX-2125, SLX-2168, SLX-2169, SLX-2170, SLX-2171, SLX-2176, and SLX-2178.

The present disclosure also provides an anti-5T4 antibody or an antigen-binding portion thereof that competes or cross-competes for binding to human 5T4 with, or binds to the same epitope of human 5T4 as, an antibody comprising:
a) a heavy chain variable region (V_{H}) comprising the amino acid sequence of SEQ ID NO:1 and a light chain variable region (V_{L}) comprising the amino acid sequence of SEQ ID NO:2;
b) a V_{H} comprising the amino acid sequence of SEQ ID NO:3 and a V_{L} Comprising the amino acid sequence of SEQ ID NO:4;
c) a V_{H} comprising the amino acid sequence of SEQ ID NO:5 and a V_{L} Comprising the amino acid sequence of SEQ ID NO:6;
d) a V_{H} comprising the amino acid sequence of SEQ ID NO:7 and a V_{L} Comprising the amino acid sequence of SEQ ID NO:8;
e) a V_{H} comprising the amino acid sequence of SEQ ID NO:7 and a V_{L} Comprising the amino acid sequence of SEQ ID NO:9;
f) a V_{H} comprising the amino acid sequence of SEQ ID NO:10 and a V_{L} Comprising the amino acid sequence of SEQ ID NO:11;
g) a V_{H} comprising the amino acid sequence of SEQ ID NO:12 and a V_{L} Comprising the amino acid sequence of SEQ ID NO:14;
h) a V_{H} comprising the amino acid sequence of SEQ ID NO:13 and a V_{L} Comprising the amino acid sequence of SEQ ID NO:14;
i) a V_{H} comprising the amino acid sequence of SEQ ID NO:10 and a V_{L} Comprising the amino acid sequence of SEQ ID NO:15;
j) a V_{H} comprising the amino acid sequence of SEQ ID NO:10 and a V_{L} Comprising the amino acid sequence of SEQ ID NO:16;
k) a V_{H} comprising the amino acid sequence of SEQ ID NO:17 and a V_{L} Comprising the amino acid sequence of SEQ ID NO:18;
l) a V_{H} comprising the amino acid sequence of SEQ ID NO:19 and a V_{L} Comprising the amino acid sequence of SEQ ID NO:20;
m) a V_{H} comprising the amino acid sequence of SEQ ID NO:21 and a V_{L} Comprising the amino acid sequence of SEQ ID NO:22;
n) a V_{H} comprising the amino acid sequence of SEQ ID NO:23 and a V_{L} Comprising the amino acid sequence of SEQ ID NO:24;
o) a V_{H} comprising the amino acid sequence of SEQ ID NO:25 and a V_{L} Comprising the amino acid sequence of SEQ ID NO:26;
p) a V_{H} comprising the amino acid sequence of SEQ ID NO:27 and a V_{L} comprising the amino acid sequence of SEQ ID NO:28;
q) a V_{H} comprising the amino acid sequence of SEQ ID NO:29 and a V_{L} Comprising the amino acid sequence of SEQ ID NO:30;
r) a V_{H} comprising the amino acid sequence of SEQ ID NO:31 and a V_{L} Comprising the amino acid sequence of SEQ ID NO:32;
s) a V_{H} comprising the amino acid sequence of SEQ ID NO:33 and a V_{L} Comprising the amino acid sequence of SEQ ID NO:34;
t) a V_{H} comprising the amino acid sequence of SEQ ID NO:35 and a V_{L} Comprising the amino acid sequence of SEQ ID NO:36;
u) a V_{H} comprising the amino acid sequence of SEQ ID NO:37 and a V_{L} Comprising the amino acid sequence of SEQ ID NO:38; or
v) a V_{H} comprising the amino acid sequence of SEQ ID NO:39 and a V_{L} Comprising the amino acid sequence of SEQ ID NO:40.
   Additionally or alternatively, an anti-5T4 antibody or antigen-binding portion thereof herein may comprise the HCDR1-3 and LCDR1-3 in said V_{H} and V_{L}, as defined by any method known in the art (e.g., by the IMGT®, Kabat, Chothia, Martin, Contact, or AHo systems, or any combination of any of these systems).

In some embodiments, the anti-5T4 antibody or antigen-binding portion thereof has a V_{H} that is at least 90% identical in sequence to any one of SEQ ID NOs:1, 3, 5, 7, 10, 12, 13, 15, 17, 19, 21, 23, 25, 27, 29, 31, 33, 35, 37, and 39, e.g., at least 92% identical, such as at least 95%, 96%, 97%, 98%, 99%, or 100% identical to the selected sequence. Additionally or alternatively, the anti-5T4 antibody or antigen-binding portion thereof may have a V_{L} that is at least 90% identical in sequence to any one of SEQ ID NOs:2, 4, 6, 8, 9, 11, 14, 16, 18, 20, 22, 24, 26, 28, 30, 32, 34, 36, 38, and 40, e.g., at least 92% identical, such as at least 95%, 96%, 97%, 98%, 99%, or 100% identical to the selected sequence. Any combination of these V_{H} and V_{L} amino acid sequences is contemplated. In certain embodiments, the V_{H} may form a heavy chain (HC) with an HC constant region (C_{H}) that is at least 90% identical in sequence to SEQ ID NO:101 or 102 (with or without the C-terminal lysine), e.g., at least 92% identical, such as at least 95%, 96%, 97%, 98%, 99%, or 100% identical to SEQ ID NO:101 or 102 (with or without the C-terminal lysine). Additionally or alternatively, the V_{L} may form a light chain (LC) with an LC constant region (C_{L}) that is at least 90% identical in sequence to SEQ ID NO:103, e.g., at least 92% identical, such as at least 95%, 96%, 97%, 98%, 99%, or 100% identical to SEQ ID NO:103.

In some embodiments, the anti-5T4 antibody or antigen-binding portion thereof may have a V_{H} that is SEQ ID NO:1, 3, 5, 7, 10, 12, 13, 15, 17, 19, 21, 23, 25, 27, 29, 31, 33, 35, 37, or 39. Additionally or alternatively, the anti-5T4 antibody or antigen-binding portion thereof may have a V_{L} that is SEQ ID NO:2, 4, 6, 8, 9, 11, 14, 16, 18, 20, 22, 24, 26, 28, 30, 32, 34, 36, 38, or 40. Any combination of these V_{H} and V_{L} amino acid sequences is contemplated. In certain embodiments, the V_{H} may form an HC with a C_{H} of SEQ ID NO:101 or 102 (with or without the terminal lysine), and/or the VL may form an LC with a C_{L} of SEQ ID NO:103.

In some embodiments, the anti-5T4 antibody or antigen-binding portion thereof has a V_{H} and a V_{L} that are at least 90% identical in sequence, e.g., at least 92% identical, such as at least 95%, 96%, 97%, 98%, or 99% identical, to the amino acid sequences of:
a) SEQ ID NOs:1 and 2, respectively;
b) SEQ ID NOs:3 and 4, respectively;
c) SEQ ID NOs:5 and 6, respectively;
d) SEQ ID NOs:7 and 8, respectively;
e) SEQ ID NOs:7 and 9, respectively;
f) SEQ ID NOs:10 and 11, respectively;
g) SEQ ID NOs:12 and 14, respectively;
h) SEQ ID NOs:13 and 14, respectively;
i) SEQ ID NOs:10 and 15, respectively;
j) SEQ ID NOs:10 and 16, respectively;
k) SEQ ID NOs:17 and 18, respectively;
l) SEQ ID NOs:19 and 20, respectively;
m) SEQ ID NOs:21 and 22, respectively;
n) SEQ ID NOs:23 and 24, respectively;
o) SEQ ID NOs:25 and 26, respectively;
p) SEQ ID NOs:27 and 28, respectively;
q) SEQ ID NOs:29 and 30, respectively;
r) SEQ ID NOs:31 and 32, respectively;
s) SEQ ID NOs:33 and 34, respectively;
t) SEQ ID NOs:35 and 36, respectively;
u) SEQ ID NOs:37 and 38, respectively; or
v) SEQ ID NOs:39 and 40, respectively.

In some embodiments, the anti-5T4 antibody or antigen-binding portion thereof has a V_{H} and a V_{L} that comprise
a) SEQ ID NOs:1 and 2, respectively;
b) SEQ ID NOs:3 and 4, respectively;
c) SEQ ID NOs:5 and 6, respectively;
d) SEQ ID NOs:7 and 8, respectively;
e) SEQ ID NOs:7 and 9, respectively;
f) SEQ ID NOs:10 and 11, respectively;
g) SEQ ID NOs:12 and 14, respectively;
h) SEQ ID NOs:13 and 14, respectively;
i) SEQ ID NOs:10 and 15, respectively;
j) SEQ ID NOs:10 and 16, respectively;
k) SEQ ID NOs:17 and 18, respectively;
l) SEQ ID NOs:19 and 20, respectively;
m) SEQ ID NOs:21 and 22, respectively;
n) SEQ ID NOs:23 and 24, respectively;
o) SEQ ID NOs:25 and 26, respectively;
p) SEQ ID NOs:27 and 28, respectively;
q) SEQ ID NOs:29 and 30, respectively;
r) SEQ ID NOs:31 and 32, respectively;
s) SEQ ID NOs:33 and 34, respectively;
t) SEQ ID NOs:35 and 36, respectively;
u) SEQ ID NOs:37 and 38, respectively; or
v) SEQ ID NOs:39 and 40, respectively.

In some embodiments, the anti-5T4 antibody has
a) an HC comprising the amino acid sequences of SEQ ID NOs:1 and 101 (optionally without the C-terminal lysine) and an LC comprising the amino acid sequences of SEQ ID NOs:2 and 103;
b) an HC comprising the amino acid sequences of SEQ ID NOs:3 and 101 (optionally without the C-terminal lysine) and an LC comprising the amino acid sequences of SEQ ID NOs:4 and 103;
c) an HC comprising the amino acid sequences of SEQ ID NOs:5 and 101 (optionally without the C-terminal lysine) and an LC comprising the amino acid sequences of SEQ ID NOs:6 and 103;
d) an HC comprising the amino acid sequences of SEQ ID NOs:7 and 101 (optionally without the C-terminal lysine) and an LC comprising the amino acid sequences of SEQ ID NOs:8 and 103;
e) an HC comprising the amino acid sequences of SEQ ID NOs:7 and 101 (optionally without the C-terminal lysine) and an LC comprising the amino acid sequences of SEQ ID NOs:9 and 103;
f) an HC comprising the amino acid sequences of SEQ ID NOs:10 and 101 (optionally without the C-terminal lysine) and an LC comprising the amino acid sequences of SEQ ID NOs:11 and 103;
g) an HC comprising the amino acid sequences of SEQ ID NOs:12 and 101 (optionally without the C-terminal lysine) and an LC comprising the amino acid sequences of SEQ ID NOs:14 and 103;
h) an HC comprising the amino acid sequences of SEQ ID NOs:13 and 101 (optionally without the C-terminal lysine) and an LC comprising the amino acid sequences of SEQ ID NOs:14 and 103;
i) an HC comprising the amino acid sequences of SEQ ID NOs:10 and 101 (optionally without the C-terminal lysine) and an LC comprising the amino acid sequences of SEQ ID NOs:15 and 103;
j) an HC comprising the amino acid sequences of SEQ ID NOs:10 and 101 (optionally without the C-terminal lysine) and an LC comprising the amino acid sequences of SEQ ID NOs:16 and 103;
k) an HC comprising the amino acid sequences of SEQ ID NOs:17 and 101 (optionally without the C-terminal lysine) and an LC comprising the amino acid sequences of SEQ ID NOs:18 and 103;
l) an HC comprising the amino acid sequences of SEQ ID NOs:19 and 101 (optionally without the C-terminal lysine) and an LC comprising the amino acid sequences of SEQ ID NOs:20 and 103;
m) an HC comprising the amino acid sequences of SEQ ID NOs:21 and 101 (optionally without the C-terminal lysine) and an LC comprising the amino acid sequences of SEQ ID NOs:22 and 103;
n) an HC comprising the amino acid sequences of SEQ ID NOs:23 and 101 (optionally without the C-terminal lysine) and an LC comprising the amino acid sequences of SEQ ID NOs:24 and 103;
o) an HC comprising the amino acid sequences of SEQ ID NOs:25 and 101 (optionally without the C-terminal lysine) and an LC comprising the amino acid sequences of SEQ ID NOs:26 and 103;
p) an HC comprising the amino acid sequences of SEQ ID NOs:27 and 101 (optionally without the C-terminal lysine) and an LC comprising the amino acid sequences of SEQ ID NOs:28 and 103;
q) an HC comprising the amino acid sequences of SEQ ID NOs:29 and 101 (optionally without the C-terminal lysine) and an LC comprising the amino acid sequences of SEQ ID NOs:30 and 103;
r) an HC comprising the amino acid sequences of SEQ ID NOs:31 and 101 (optionally without the C-terminal lysine) and an LC comprising the amino acid sequences of SEQ ID NOs:32 and 103;
s) an HC comprising the amino acid sequences of SEQ ID NOs:33 and 101 (optionally without the C-terminal lysine) and an LC comprising the amino acid sequences of SEQ ID NOs:34 and 103;
t) an HC comprising the amino acid sequences of SEQ ID NOs:35 and 101 (optionally without the C-terminal lysine) and an LC comprising the amino acid sequences of SEQ ID NOs:36 and 103;
u) an HC comprising the amino acid sequences of SEQ ID NOs:37 and 101 (optionally without the C-terminal lysine) and an LC comprising the amino acid sequences of SEQ ID NOs:38 and 103; or
v) an HC comprising the amino acid sequences of SEQ ID NOs:39 and 101 (optionally without the C-terminal lysine) and an LC comprising the amino acid sequences of SEQ ID NOs:40 and 103.

In some embodiments, the anti-5T4 antibody or antigen-binding portion thereof has
a) an HC comprising the amino acid sequences of SEQ ID NOs:1 and 102 (optionally without the C-terminal lysine) and an LC comprising the amino acid sequences of SEQ ID NOs:2 and 103;
b) an HC comprising the amino acid sequences of SEQ ID NOs:3 and 102 (optionally without the C-terminal lysine) and an LC comprising the amino acid sequences of SEQ ID NOs:4 and 103;
c) an HC comprising the amino acid sequences of SEQ ID NOs:5 and 102 (optionally without the C-terminal lysine) and an LC comprising the amino acid sequences of SEQ ID NOs:6 and 103;
d) an HC comprising the amino acid sequences of SEQ ID NOs:7 and 102 (optionally without the C-terminal lysine) and an LC comprising the amino acid sequences of SEQ ID NOs:8 and 103;
e) an HC comprising the amino acid sequences of SEQ ID NOs:7 and 102 (optionally without the C-terminal lysine) and an LC comprising the amino acid sequences of SEQ ID NOs:9 and 103;
f) an HC comprising the amino acid sequences of SEQ ID NOs:10 and 102 (optionally without the C-terminal lysine) and an LC comprising the amino acid sequences of SEQ ID NOs:11 and 103;
g) an HC comprising the amino acid sequences of SEQ ID NOs:12 and 102 (optionally without the C-terminal lysine) and an LC comprising the amino acid sequences of SEQ ID NOs:14 and 103;
h) an HC comprising the amino acid sequences of SEQ ID NOs:13 and 102 (optionally without the C-terminal lysine) and an LC comprising the amino acid sequences of SEQ ID NOs:14 and 103;
i) an HC comprising the amino acid sequences of SEQ ID NOs:10 and 102 (optionally without the C-terminal lysine) and an LC comprising the amino acid sequences of SEQ ID NOs:15 and 103;
j) an HC comprising the amino acid sequences of SEQ ID NOs:10 and 102 (optionally without the C-terminal lysine) and an LC comprising the amino acid sequences of SEQ ID NOs:16 and 103;
k) an HC comprising the amino acid sequences of SEQ ID NOs:17 and 102 (optionally without the C-terminal lysine) and an LC comprising the amino acid sequences of SEQ ID NOs:18 and 103;
l) an HC comprising the amino acid sequences of SEQ ID NOs:19 and 102 (optionally without the C-terminal lysine) and an LC comprising the amino acid sequences of SEQ ID NOs:20 and 103;
m) an HC comprising the amino acid sequences of SEQ ID NOs:21 and 102 (optionally without the C-terminal lysine) and an LC comprising the amino acid sequences of SEQ ID NOs:22 and 103;
n) an HC comprising the amino acid sequences of SEQ ID NOs:23 and 102 (optionally without the C-terminal lysine) and an LC comprising the amino acid sequences of SEQ ID NOs:24 and 103;
o) an HC comprising the amino acid sequences of SEQ ID NOs:25 and 102 (optionally without the C-terminal lysine) and an LC comprising the amino acid sequences of SEQ ID NOs:26 and 103;
p) an HC comprising the amino acid sequences of SEQ ID NOs:27 and 102 (optionally without the C-terminal lysine) and an LC comprising the amino acid sequences of SEQ ID NOs:28 and 103;
q) an HC comprising the amino acid sequences of SEQ ID NOs:29 and 102 (optionally without the C-terminal lysine) and an LC comprising the amino acid sequences of SEQ ID NOs:30 and 103;
r) an HC comprising the amino acid sequences of SEQ ID NOs:31 and 102 (optionally without the C-terminal lysine) and an LC comprising the amino acid sequences of SEQ ID NOs:32 and 103;
s) an HC comprising the amino acid sequences of SEQ ID NOs:33 and 102 (optionally without the C-terminal lysine) and an LC comprising the amino acid sequences of SEQ ID NOs:34 and 103;
t) an HC comprising the amino acid sequences of SEQ ID NOs:35 and 102 (optionally without the C-terminal lysine) and an LC comprising the amino acid sequences of SEQ ID NOs:36 and 103;
u) an HC comprising the amino acid sequences of SEQ ID NOs:37 and 102 (optionally without the C-terminal lysine) and an LC comprising the amino acid sequences of SEQ ID NOs:38 and 103; or
v) an HC comprising the amino acid sequences of SEQ ID NOs:39 and 102 (optionally without the C-terminal lysine) and an LC comprising the amino acid sequences of SEQ ID NOs:40 and 103.

In some embodiments, the anti-5T4 antibody has a heavy chain CDR1 (HCDR1) that is or is at least 90% identical in sequence to SEQ ID NO:47, e.g. at least 92% identical, such as at least 95%, 96%, 97%, 98%, or 99% identical.

In some embodiments, the anti-5T4 antibody has a heavy chain CDR2 (HCDR2) that is or is at least 90% identical in sequence to SEQ ID NO:52, e.g. at least 92% identical, such as at least 95%, 96%, 97%, 98%, or 99% identical.

In some embodiments, the anti-5T4 antibody has a heavy chain CDR3 (HCDR3) that is or is at least 90% identical in sequence to SEQ ID NO:57, e.g. at least 92% identical, such as at least 95%, 96%, 97%, 98% or 99% identical.

In some embodiments, the anti-5T4 antibody has a light chain CDR1 (LCDR1) that is or is at least 90% identical in sequence to SEQ ID NO:60, e.g. at least 92% identical, such as at least 95%, 96%, 97%, 98%, or 99% identical.

In some embodiments, the anti-5T4 antibody has a light chain CDR2 (LCDR2) that is or is at least 90% identical in sequence to SEQ ID NO:63, e.g. at least 92% identical, such as at least 95%, 96%, 97%, 98%, or 99% identical.

In some embodiments, the anti-5T4 antibody has a light chain CDR3 (LCDR3) that is or is at least 90% identical in sequence to SEQ ID NO:66, e.g. at least 92% identical, such as at least 95%, 96%, 97%, 98%, or 99% identical.

Any combination of the above HCDR1, HCDR2, HCDR3, LCDR1, LCDR2, and LCDR3 sequences is also contemplated.

In some embodiments, the anti-5T4 antibody has a heavy chain CDR1 (HCDR1) that is or is at least 90% identical in sequence to SEQ ID NO:68 or 73, e.g. at least 92% identical, such as at least 95%, 96%, 97%, 98%, or 99% identical.

In some embodiments, the anti-5T4 antibody has a heavy chain CDR2 (HCDR2) that is or is at least 90% identical in sequence to SEQ ID NO:74 or 75, e.g. at least 92% identical, such as at least 95%, 96%, 97%, 98%, or 99% identical.

In some embodiments, the anti-5T4 antibody has a heavy chain CDR3 (HCDR3) that is or is at least 90% identical in sequence to SEQ ID NO:79 or 80, e.g. at least 92% identical, such as at least 95%, 96%, 97%, 98% or 99% identical.

In some embodiments, the anti-5T4 antibody has a light chain CDR1 (LCDR1) that is or is at least 90% identical in sequence to SEQ ID NO:82 or 83, e.g. at least 92% identical, such as at least 95%, 96%, 97%, 98%, or 99% identical.

In some embodiments, the anti-5T4 antibody has a light chain CDR2 (LCDR2) that is or is at least 90% identical in sequence to SEQ ID NO:85 or 86, e.g. at least 92% identical, such as at least 95%, 96%, 97%, 98%, or 99% identical.

In some embodiments, the anti-5T4 antibody has a light chain CDR3 (LCDR3) that is or is at least 90% identical in sequence to SEQ ID NO:88, e.g. at least 92% identical, such as at least 95%, 96%, 97%, 98%, or 99% identical.

Any combination of the above HCDR1, HCDR2, HCDR3, LCDR1, LCDR2, and LCDR3 sequences is also contemplated.

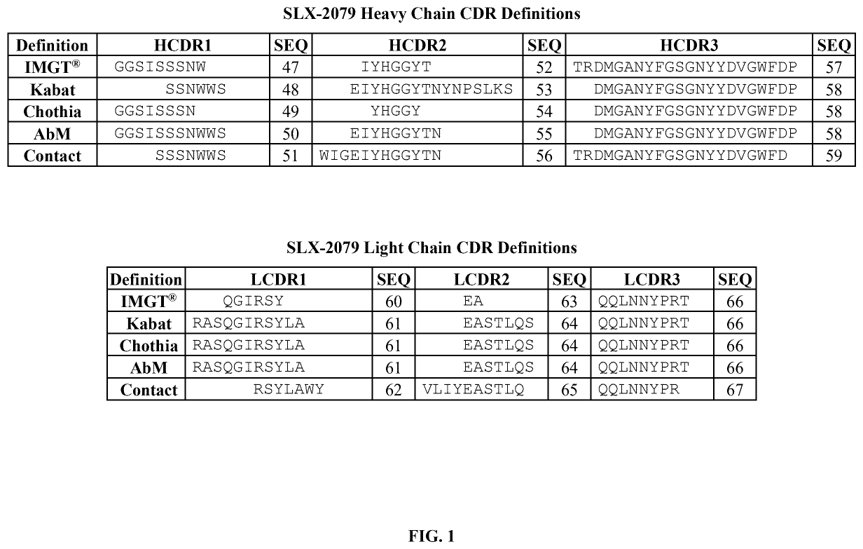

In some embodiments, the anti-5T4 antibody or antigen-binding portion of the present disclosure comprises the HCDR1-3 and LCDR1-3 amino acid sequences of any one of the anti-5T4 antibodies exemplified herein. The assignment of CDR regions may be in accordance with any method known in the art, such as IMGT®, Kabat, Chothia, Martin, Contact, or AHo definitions, or any combination of any of these definitions (Kabat plus Chothia, for example). Examples of CDR definitions under different systems are shown for SLX-2079 in FIG. 1:

In certain embodiments,
in any of the SLX-2079 HCDR2 sequences, the residue corresponding to the G at position 55 of SEQ ID NO:1 (G54 by Kabat numbering) may be substituted, e.g., with A, M, or R,
in any of the SLX-2079 HCDR3 sequences, the residue corresponding to the A at position 102 of SEQ ID NO:1 (A98 by Kabat numbering) may be substituted, e.g., with I or V, and/or the residue corresponding to the F at position 105 of SEQ ID NO:1 (F100a by Kabat numbering) may be substituted, e.g., with W or Y,
in any of the SLX-2079 LCDR1 sequences, the residue corresponding to the Q at position 27 of SEQ ID NO:2 (Q27 by Kabat numbering) may be substituted, e.g., with W or Y, or
any combination of the above substitutions.
   For example, the antibody or antigen-binding portion with HCDR1-3 and LCDR1-3 from SLX-2079 (defined by any system herein) may comprise
an SLX-2079 HCDR3 with a F100aY substitution,
an SLX-2079 HCDR2 with a G54A substitution and an SLX-2079 HCDR3 with A981 and F100aY substitutions, or
an SLX-2079 HCDR2 with a G54M substitution and an SLX-2079 HCDR3 with an F100aY substitution,
   wherein the residues are numbered according to Kabat.

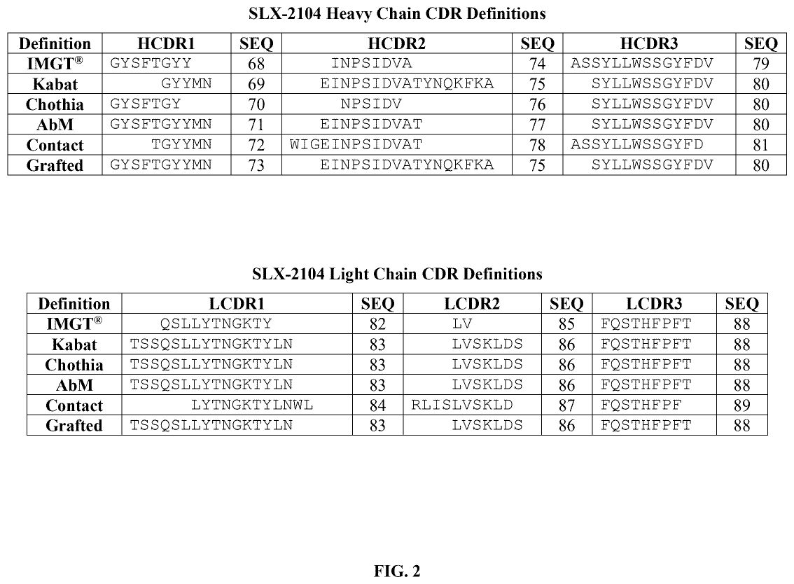

Examples of CDR definitions under different systems are shown for SLX-2104 in FIG. 2.

In certain embodiments, in any of the SLX-2104 LCDR1 sequences, the residue corresponding to the N at position 33 of SEQ ID NO:4 (N28 by Kabat numbering) may be substituted, e.g., with Q or S, and/or the residue corresponding to the G at position 34 of SEQ ID NO:4 (G29 by Kabat numbering) may be substituted, e.g., with A.

For example, the antibody or antigen-binding portion with HCDR1-3 and LCDR1-3 from SLX-2104 (defined by any system herein) may comprise
an SLX-2104 LCDR1 with a N28Q substitution,
an SLX-2104 LCDR1 with a N28S substitution, or
an SLX-2104 LCDR1 with a G29A substitution,
   wherein the residues are numbered according to Kabat.

Thus, for example, the SLX-2079 IMGT®-defined HCDR1-3 and LCDR1-3 sequences of SEQ ID NOs:47, 52, 57, 60, 63, and 66, respectively, may be replaced in any embodiment described herein by
SEQ ID NOs:48, 53, 58, 61, 64, and 66, respectively;
SEQ ID NOs:49, 54, 58, 61, 64, and 66, respectively;
SEQ ID NOs:50, 55, 58, 61, 64, and 66, respectively; or
SEQ ID NOs:51, 56, 59, 62, 65, and 67, respectively.

Similarly, for example, the SLX-2104 IMGT®-defined HCDR1-3 and LCDR1-3 sequences of SEQ ID NOs:68, 74, 79, 82, 85, and 88, respectively, may be replaced in any embodiment described herein by
SEQ ID NOs:69, 75, 80, 83, 86, and 88, respectively;
SEQ ID NOs:70, 76, 80, 83, 86, and 88, respectively;
SEQ ID NOs:71, 77, 80, 83, 86, and 88, respectively;
SEQ ID NOs:72, 78, 81, 84, 87, or 89, respectively or
SEQ ID NOs:73, 75, 80, 83, 86, and 88, respectively.

Also contemplated is a set of SLX-2079 or SLX-2104 CDRs wherein each of HCDR1, HCDR2, HCDR3, LCDR1, LCDR2, and LCDR3 may individually be specified according to any of the methods for defining SLX-2079 or SLX-2104 CDRs as shown above (e.g., HCDR1 specified by the Kabat definition, HCDR2 specified by the Chothia definition, etc.). For example, in some embodiments, the anti-5T4 antibody or antigen-binding portion thereof comprises
an HCDR1 comprising SEQ ID NO: 47, 48, 49, 50, or 51;
an HCDR2 comprising SEQ ID NO: 52, 53, 54, 55, or 56;
an HCDR3 comprising SEQ ID NO: 57, 58, or 59;
an LCDR1 comprising SEQ ID NO: 60, 61, or 62;
an LCDR2 comprising SEQ ID NO: 63, 64, or 65; and/or an LCDR3 comprising SEQ ID NO: 66 or 67.
   In other embodiments, the antibody or antigen-binding portion thereof comprises
an HCDR1 comprising SEQ ID NO: 68, 69, 70, 71, 72, or 73;
an HCDR2 comprising SEQ ID NO: 74, 75, 76, 77, or 78;
an HCDR3 comprising SEQ ID NO: 79, 80, or 81;
an LCDR1 comprising SEQ ID NO: 82, 83, or 84;
an LCDR2 comprising SEQ ID NO: 85, 86, or 87; and/or
an LCDR3 comprising SEQ ID NO: 88 or 89.

The same means for defining SLX-2079 and SLX-2104 CDRs are contemplated for any of the exemplified antibodies herein.

In some embodiments, the anti-5T4 antibody or antigen-binding portion thereof has:
an HCDR1 selected from SEQ ID NOs:47-51;
an HCDR2 selected from SEQ ID NOs:52-56 and 90-92;
an HCDR3 selected from SEQ ID NOs:57-59 and 93-97;
an LCDR1 selected from SEQ ID NOs:60-62 and 98-100;
an LCDR2 selected from SEQ ID NOs:63-65; and
an LCDR3 selected from SEQ ID NOs:66 and 67.

In some embodiments, the anti-5T4 antibody or antigen-binding portion thereof has HCDR1-3 and LCDR1-3 comprising:
SEQ ID NOs:47, 52, 57, 60, 63, and 66, respectively;
SEQ ID NOs:47, 52, 95, 60, 63, and 66, respectively;
SEQ ID NOs:47, 90, 97, 60, 63, and 66, respectively; or
SEQ ID NOs:47, 91, 95, 60, 63, and 66, respectively.

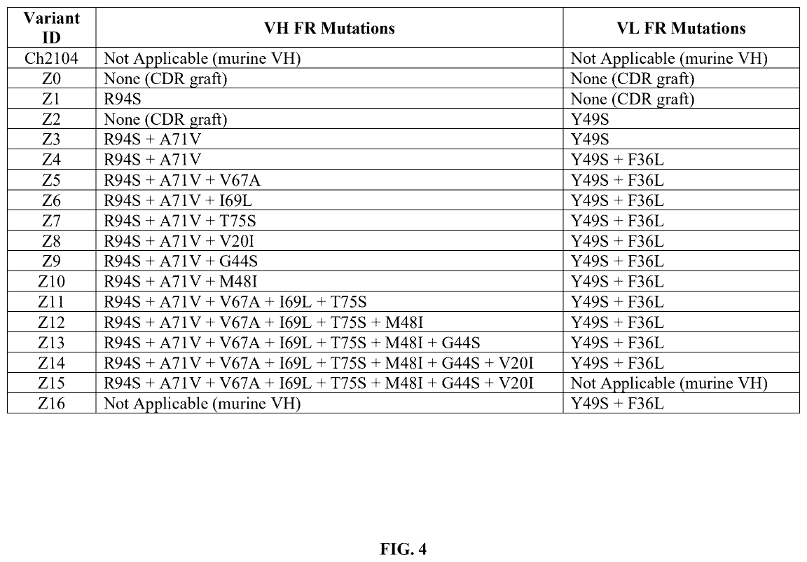

In some embodiments, the anti-5T4 antibody or antigen-binding portion has HCDR1-3 and LCDR1-3 comprising:
SEQ ID NOs:68, 74, 79, 82, 85, and 88, respectively; or
SEQ ID NOs:73, 75, 80, 83, 86, and 88, respectively.
   In certain embodiments, the HCDR1-3 are in a V_{H} that comprises framework regions derived from a human IGHV1-69*02 germline gene. The V_{H} may comprise one or more back mutations at positions selected from V20, G44, M48, V67, 169, A71, T75, R94, or any combination thereof (e.g., V20I, G44S, M48I, V67A, I69L, A71V, T75S, R94S, or any combination thereof, such as a combination found in FIG. 4), wherein the numbering of the residues is according to Kabat. In certain embodiments, the LCDR1-3 are in a V_{L} that comprises framework regions derived from a human IGKV2-30*01 germline gene. The V_{L} may comprise one or more back mutations at positions selected from F36, Y49, or both (e.g., F36L, Y49S, or both, such as a combination found in FIG. 4), wherein the numbering of the residues is according to Kabat. Any combination of a V_{H} and V_{L} as described above is also contemplated (such as a combination shown for any one of Z1-Z16 in FIG. 4).

In some embodiments, the anti-5T4 antibody or antigen-binding portion thereof of the present disclosure may have a V_{H} that is at least 90% identical in sequence to any one of SEQ ID NOs:1, 35, 37, and 39, e.g. at least 92% identical, such as at least 95%, 96%, 97%, 98%, or 99% identical to any one of SEQ ID NOs:1, 35, 37, and 39. Additionally or alternatively, the anti-5T4 antibody or antigen-binding portion thereof may have a V_{L} that is at least 90% identical in sequence to any one of SEQ ID NOs:2, 36, 38, and 40, e.g. at least 92% identical, such as at least 95%, 96%, 97%, 98%, or 99% identical to any one of SEQ ID NOs:2, 36, 38, and 40. Any combination of these V_{H} and V_{L} amino acid sequences is contemplated. In some embodiments, the V_{H} may be any one of SEQ ID NOs:1, 35, 37, and 39 and the V_{L} may be any one of SEQ ID NOs:2, 36, 38, and 40.

In some embodiments, the anti-5T4 antibody may have a heavy chain (HC) amino acid sequence comprising a V_{H} that is or is at least 80%, 85%, 90%, 91%, 92%, 93%, 94%, 95%, 96%, 97%, 98%, or 99% identical to the amino acid sequence of SEQ ID NO:1, 35, 37, or 39 and a heavy chain constant domain (C_{H}) that is or is at least 80%, 85%, 90%, 91%, 92%, 93%, 94%, 95%, 96%, 97%, 98%, or 99% identical to the amino acid sequence of SEQ ID NO:101 or 102, optionally without the C-terminal lysine. Additionally or alternatively, the anti-5T4 antibody may have a light chain (LC) amino acid sequence comprising a V_{L} that is or is at least 80%, 85%, 90%, 91%, 92%, 93%, 94%, 95%, 96%, 97%, 98%, or 99% identical to the amino acid sequence of SEQ ID NO:2, 36, 38, or 40 and a light chain constant domain (C_{L}) that is or is at least 80%, 85%, 90%, 91%, 92%, 93%, 94%, 95%, 96%, 97%, 98%, or 99% identical to the amino acid sequence of SEQ ID NO:103. Any combination of these HC and LC amino acid sequences is also contemplated. In some embodiments, the HC may comprise SEQ ID NOs:1 and 101, 35 and 101, 37 and 101, or 39 and 101 (wherein SEQ ID NO: 101 optionally may lack the C-terminal lysine) and the LC may comprise SEQ ID NOs:2 and 103, 36 and 103, 38 and 103, or 40 and 103. In some embodiments, the HC may comprise SEQ ID NOs:1 and 102, 35 and 102, 37 and 102, or 39 and 102 (wherein SEQ ID NO: 102 optionally may lack the C-terminal lysine) and the LC may comprise SEQ ID NOs:2 and 103, 36 and 103, 38 and 103, or 40 and 103.

In some embodiments, the anti-5T4 antibody or antigen-binding portion thereof may have a V_{H} that is at least 90% identical in sequence to any one of SEQ ID NOs:3, 29, 31, and 33, e.g. at least 92% identical, such as at least 95%, 96%, 97%, 98%, or 99% identical to any one of SEQ ID NOs:3, 29, 31, and 33. Additionally or alternatively, the anti-5T4 antibody or antigen-binding portion thereof may have a V_{L} that is at least 90% identical in sequence to any one of SEQ ID NOs:4, 30, 32, and 34, e.g. at least 92% identical, such as at least 95%, 96%, 97%, 98%, or 99% identical to any one of SEQ ID NOs:4, 30, 32, and 34; in certain embodiments, differences from the selected sequence comprise, or consist of, substitution of N28 with Q or S and/or substitution of G29 with A (residues numbered according to Kabat). Any combination of these V_{H} and V_{L} amino acid sequences is contemplated. In some embodiments, the V_{H} may be any one of SEQ ID NOs:3, 29, 31, and 33 and the V_{L} may be any one of SEQ ID NOs:4, 30, 32, and 34.

In some embodiments, the anti-5T4 antibody or antigen-binding portion thereof may have a V_{H} that is at least 90% identical in sequence to any one of SEQ ID NOs:29, 31, and 33, e.g. at least 92% identical, such as at least 95%, 96%, 97%, 98%, or 99% identical to any one of SEQ ID NOs:29, 31, and 33. Additionally or alternatively, the anti-5T4 antibody or antigen-binding portion thereof may have a V_{L} that is at least 90% identical in sequence to any one of SEQ ID NOs:30, 32, and 34, e.g. at least 92% identical, such as at least 95%, 96%, 97%, 98%, or 99% identical to any one of SEQ ID NOs:30, 32, and 34; in certain embodiments, differences from the selected sequence comprise, or consist of, substitution of N28 with Q or S and/or substitution of G29 with A (residues numbered according to Kabat). Any combination of these V_{H} and V_{L} amino acid sequences is contemplated. In some embodiments, the V_{H} may be any one of SEQ ID NOs:29, 31, and 33 and the V_{L} may be any one of SEQ ID NOs:30, 32, and 34.

In some embodiments, the anti-5T4 antibody has a heavy chain (HC) amino acid sequence comprising a V_{H} that is or is at least 80%, 85%, 90%, 91%, 92%, 93%, 94%, 95%, 96%, 97%, 98%, or 99% identical to the amino acid sequence of SEQ ID NO:3, 29, 31, or 33 (in certain embodiments, differences from the selected sequence comprise, or consist of, substitution of N28 with Q or S and/or substitution of G29 with A, residues numbered according to Kabat) and a C_{H} that is or is at least 80%, 85%, 90%, 91%, 92%, 93%, 94%, 95%, 96%, 97%, 98%, or 99% identical to the amino acid sequence of SEQ ID NO:101 or 102, optionally without the C-terminal lysine. Additionally or alternatively, the anti-5T4 antibody may have a light chain (LC) amino acid sequence comprising a V_{L} that is or is at least 80%, 85%, 90%, 91%, 92%, 93%, 94%, 95%, 96%, 97%, 98%, or 99% identical to the amino acid sequence of SEQ ID NO:4, 30, 32, or 34 and a C_{L} that is or is at least 80%, 85%, 90%, 91%, 92%, 93%, 94%, 95%, 96%, 97%, 98%, or 99% identical to the amino acid sequence of SEQ ID NO:103. Any combination of these HC and LC amino acid sequences is also contemplated. In some embodiments, the HC may comprise SEQ ID NOs:3 and 101, 29 and 101, 31 and 101, or 33 and 101 (wherein SEQ ID NO: 101 optionally may lack the C-terminal lysine) and the LC may comprise SEQ ID NOs:4 and 103, 30 and 103, 32 and 103, or 34 and 103. In some embodiments, the HC may comprise SEQ ID NOs:3 and 102, 29 and 102, 31 and 102, or 33 and 102 (wherein SEQ ID NO: 102 optionally may lack the C-terminal lysine) and the LC may comprise SEQ ID NOs:4 and 103, 30 and 103, 32 and 103, or 34 and 103.

In some embodiments, the anti-5T4 antibody has a heavy chain (HC) amino acid sequence comprising a V_{H} that is or is at least 80%, 85%, 90%, 91%, 92%, 93%, 94%, 95%, 96%, 97%, 98%, or 99% identical to the amino acid sequence of SEQ ID NO:29, 31, or 33 (in certain embodiments, differences from the selected sequence comprise, or consist of, substitution of N28 with Q or S and/or substitution of G29 with A, residues numbered according to Kabat) and a C_{H} that is or is at least 80%, 85%, 90%, 91%, 92%, 93%, 94%, 95%, 96%, 97%, 98%, or 99% identical to the amino acid sequence of SEQ ID NO:101 or 102, optionally without the C-terminal lysine. Additionally or alternatively, the anti-5T4 antibody may have a light chain (LC) amino acid sequence comprising a V_{L} that is or is at least 80%, 85%, 90%, 91%, 92%, 93%, 94%, 95%, 96%, 97%, 98%, or 99% identical to the amino acid sequence of SEQ ID NO:30, 32, or 34 and a C_{L} that is or is at least 80%, 85%, 90%, 91%, 92%, 93%, 94%, 95%, 96%, 97%, 98%, or 99% identical to the amino acid sequence of SEQ ID NO:103. Any combination of these HC and LC amino acid sequences is also contemplated. In some embodiments, the HC may comprise SEQ ID NOs:29 and 101, 31 and 101, or 33 and 101 (wherein SEQ ID NO: 101 optionally may lack the C-terminal lysine) and the LC may comprise SEQ ID NOs:30 and 103, 32 and 103, or 34 and 103. In some embodiments, the HC may comprise SEQ ID NOs:29 and 102, 31 and 102, or 33 and 102 (wherein SEQ ID NO: 102 optionally may lack the C-terminal lysine) and the LC may comprise SEQ ID NOs:30 and 103, 32 and 103, or 34 and 103.

In some embodiments, the anti-5T4 antibody of the present disclosure comprises an HC comprising SEQ ID NOs:1 and 102 (optionally without the C-terminal lysine) and an LC comprising SEQ ID NOs:2 and 103.

In some embodiments, the anti-5T4 antibody of the present disclosure comprises an HC comprising SEQ ID NOs:3 and 102 (optionally without the C-terminal lysine) and an LC comprising SEQ ID NOs:4 and 103.

In some embodiments, the anti-5T4 antibody of the present disclosure comprises an HC comprising SEQ ID NOs:29 and 102 (optionally without the C-terminal lysine) and an LC comprising SEQ ID NOs:30 and 103.

In some embodiments, the anti-5T4 antibody of the present disclosure comprises an HC comprising SEQ ID NOs:31 and 102 (optionally without the C-terminal lysine) and an LC comprising SEQ ID NOs:32 and 103.

In some embodiments, the anti-5T4 antibody of the present disclosure comprises an HC comprising SEQ ID NOs:33 and 102 (optionally without the C-terminal lysine) and an LC comprising SEQ ID NOs:34 and 103.

The anti-5T4 antibody of the present disclosure can be an IgG, an IgM, an IgE, an IgA, or an IgD molecule. The heavy-chain constant regions (Fc) that correspond to the different classes of immunoglobulins can be γ, μ, ε, α, or δ, respectively. In certain embodiments, the anti-5T4 antibody is of the IgG isotype, e.g., of IgG subclass IgG₁, IgG₂, IgG3 or IgG4. In some embodiments, the antibody is of the isotype subclass IgG₁. The light chain constant regions of the antibody can be kappa (κ) or lambda (λ).

In some embodiments, an anti-5T4 antibody or antigen-binding portion herein may bind to human 5T4; human and cynomolgus 5T4; or human, cynomolgus, and mouse 5T4. In certain embodiments, the antibody or portion may bind to human 5T4 with a K_{D} of 1500, 1000, 900, 800, 700, 600, 500, 400, 300, 200, 100, 90, 80, 70, 60, 50, 40, 30, 20, 10, 5, 1, 0.5, 0.1, 0.05, or 0.01 nM or less (for example, 600 nM or less), e.g., as measured by surface plasmon resonance. In certain additional or alternative embodiments, the antibody or portion may bind to cynomolgus 5T4 with a K_{D} of 1500, 1000, 900, 800, 700, 600, 500, 400, 300, 200, 100, 90, 80, 70, 60, 50, 40, 30, 20, 10, 5, 1, 0.5, 0.1, 0.05, or 0.01 nM or less, e.g., as measured by surface plasmon resonance.

In some embodiments, the antibody or portion may bind to human 5T4 expressed on MCF7 cells with an EC₅₀ of 100, 50, 20, 18, 15, 12, 10, 9, 8, 7, 6, 5, 4, 3, 2, 1, 0.5, 0.3, or 0.1 nM or less.

The present disclosure also contemplates an anti-5T4 antibody or antigen-binding portion with any combination of the above properties.

The present disclosure also encompasses 5T4-binding proteins that are variants of those specifically described herein. For example, the variants may comprise mutations that confer desired properties to the proteins, e.g., increased half-life, altered (e.g., reduced) immunogenicity in humans, and/or engineered sites for covalent or non-covalent binding to another molecule (e.g., a cytotoxic, radioactive, or detectable agent). These mutations may be introduced in the FRs, the CDRs, or other parts of the proteins (e.g., the Ig constant regions, if the proteins contain such regions).

In some embodiments, an anti-5T4 antibody herein may comprise at least one mutation in the Fc region. A number of different Fc mutations are known, where these mutations alter, e.g., the antibody's effector functions or half-life. For example, in some embodiments, the constant region of an antibody (e.g., an anti-5T4 antibody herein) may comprise mutations that improve the therapeutic potential of the antibody, such as mutations that reduce or eliminate effector functions of the antibody. An antibody may comprise, for instance, a human IgG₁ constant region with the L235E mutation, “LALA” mutations (L234A/L235A), “LALAGA” mutations (L234A/L235A/G237A), “LALAGR” mutations (L234A/L235A/G236R), “LALAPA” mutations (L234A/L235A/P329A), or “LALAPG” mutations (L234A/L235A/P329G) (Eu numbering is used when referring to constant region mutations, unless otherwise indicated). In some embodiments, the constant region may comprise L234F/L235E/P331S (“FES”), L234F/L235Q/K322Q (“FQQ”), A330S/P331S, L234A/G237A, L234A/L235A/G237A, L234A/L235A/G237A/P238S/H268A/A330S/P330S, L234A/L235E, G236R/L328R, or L234A/L235A/K322A mutations. The present disclosure contemplates, for instance, an anti-5T4 antibody herein with any mutation that reduces or eliminates effector functions as described in Wilkinson et al., PLoS One (2021) 16(12):e0260954. Additionally or alternatively, the human IgG₁ constant region may comprise, for instance, a human heavy chain constant region with the mutation P329A. In some embodiments, the antibody may comprise a human IgG4 constant region with the mutation L235E and/or the mutation S228P. An IgG constant region may comprise mutations that improve the serum half-life of the antibody (e.g., the “YTE” mutation) and/or improve manufacturing and yield of the antibody.

In certain embodiments, the anti-5T4 antibody may comprise, e.g., L234A, L235A, and/or P329A mutations (Eu numbering), wherein the mutations may appear alone or in any combination. In particular embodiments, the anti-5T4 antibody may comprise an Fc region with all three mutations.

The present disclosure also encompasses 5T4-binding proteins that are bispecific or multispecific, comprising an anti-5T4 antibody or an antigen-binding portion thereof herein.

### II. Making of 5T4-Binding Proteins

The 5T4-binding protein of the present disclosure may be produced recombinantly using isolated nucleic acid molecules such as expression constructs. The coding sequences for each polypeptide chain may be cloned into a single vector or cloned into separate vectors (e.g., a pair or set of vectors). The proteins may be produced in host cells, e.g., mammalian host cells, using appropriate expression constructs. Mammalian cell lines available as hosts for expression include, without limitation, Chinese hamster ovary (CHO) cells, NS0 cells, SP2 cells, HEK-293T cells, 293 Freestyle cells (Invitrogen), NIH-3T3 cells, HeLa cells, baby hamster kidney (BHK) cells, African green monkey kidney cells (COS), human hepatocellular carcinoma cells (e.g., Hep G2), and A549 cells. Other cell lines that may be used are insect cell lines, such as Sf9 or Sf21 cells, and yeast cell lines. In certain embodiments, the cell lines are not derived from a human embryo. Cell lines may be selected based on their expression levels.

Host cells used to produce the 5T4-binding proteins are “recombinant host cells.” A “recombinant host cell” (or simply “host cell”), as used herein, means a cell into which a recombinant expression construct has been introduced. By definition, a recombinant host cell does not occur in nature. A protein produced from a recombinant host cell is a recombinant protein.

The 5T4-binding protein may be isolated and purified from the host cell culture using well known methods, including but not limited to centrifugation; ultracentrifugation; protein A, protein G, protein A/G, or protein L purification; purification with affinity tags such as poly-histidine (e.g., using nickel (such as Ni-NTA) or cobalt); and/or ion exchange chromatography.

### III. Pharmaceutical Compositions

Another aspect of the present disclosure is a pharmaceutical composition comprising as an active ingredient (or as the sole active ingredient) the 5T4-binding protein of the present disclosure. The pharmaceutical composition may additionally comprise a pharmaceutically acceptable excipient. A “pharmaceutically acceptable excipient” may include appropriate solvents, dispersion media, antibacterial and antifungal agents, isotonic agents, and the like. Examples of pharmaceutically acceptable excipients are water and saline (e.g., phosphate-buffered saline).

The pharmaceutical compositions may be used to treat 5T4-associated diseases, e.g., diseases where 5T4 is expressed or overexpressed. In some embodiments, the pharmaceutical compositions are intended for amelioration, prevention, and/or treatment of a 5T4-related disorder and/or cancer. As used herein, a 5T4-related or -mediated disorder refers to a disorder, disease or condition that improves, or slows down in its progression, by modulation of 5T4 activity. In some embodiments, the pharmaceutical compositions are used to treat a 5T4-expressing cancer. In some embodiments, the pharmaceutical compositions are used to treat a 5T4-positive cancer.

In certain embodiments, the compositions are intended for amelioration, prevention, and/or treatment of cancer originating in tissues such as skin, lung, intestine, colon, ovary, brain, prostate, kidney, bones, soft tissues, the hematopoietic system, head and neck, liver, bladder, breast, stomach, cervical, endometrial, uterus, gastric, esophageal, and pancreas. The cancer may be a solid tumor or a hematopoietic cancer.

In some embodiments, the cancer is selected from melanoma, skin basal cell cancer, glioblastoma, glioma, gliosarcoma, astrocytoma, meningioma, neuroblastoma, adrenocortical cancer, head and neck cancer (e.g., cancer of the head, neck, nasal cavity, paranasal sinuses, nasopharynx, oral cavity, oropharynx, larynx, hypopharynx, and/or salivary glands, and paragangliomas), oral cancer, salivary gland cancer, nasopharyngeal cancer, breast cancer (e.g., triple negative breast cancer), lung cancer (e.g., non-small cell lung cancer (NSCLC), small cell lung cancer, or squamous cell lung cancer), esophageal cancer, gastroesophageal junction cancer, gastric cancer, gastrointestinal cancer, primary peritoneal cancer, liver cancer, hepatocellular carcinoma, gallbladder cancer, biliary tract cancer, cholangiocarcinoma, colon cancer, rectal cancer, colorectal carcinoma, ovarian cancer, fallopian tube cancer, bladder cancer, upper urinary tract cancer, urothelial cancer, renal cell carcinoma, kidney cancer, genitourinary cancer, cervical cancer, testicular cancer, prostate cancer, fibrosarcoma, liposarcoma, rhabdomyosarcoma (e.g., embryonal rhabdomyosarcoma), leiomyosarcoma, neurofibrosarcoma, synovial sarcoma, liposarcoma, alveolar soft part sarcoma, osteosarcoma, histiocytoma (e.g., malignant fibrous histiocytoma), pancreatic cancer, endometrial cancer, cancer of the appendix, thyroid cancer, advanced Merkel cell cancer, multiple myeloma, sarcomas, choriocarcinoma, leukemia (e.g., erythroleukemia, acute lymphoblastic leukemia, acute monocytic leukemia, acute promyelocytic leukemia, acute myeloid leukemia, acute myelogenous leukemia, chronic myeloid leukemia, chronic myelogenous leukemia, chronic lymphocytic leukemia, acute lymphoblastic leukemia, or mast cell leukemia), lymphoma (e.g., small lymphocytic lymphoma, Burkitt's lymphoma, Hodgkin's lymphoma, non-Hodgkin's lymphoma, diffuse large B cell lymphoma, lymphoplasmacytoid lymphoma, mucosa-associated lymphoid tissue lymphoma, mantle cell lymphoma, T-cell anaplastic large cell lymphoma, follicular lymphoma, monocytic lymphoma, or HTLV-associated T cell leukemia/lymphoma), and mesothelioma. In certain embodiments, the cancer is selected from the group consisting of head and neck cancer, bone cancer (e.g., osteosarcoma), Ewing sarcoma, squamous cell carcinoma, lung cancer (e.g., non-small cell lung cancer or small cell lung cancer), kidney cancer, urethral cancer, colorectal cancer, prostate cancer, glioblastoma multiforme, ovarian cancer, cervical cancer, pancreatic cancer, breast cancer (e.g., triple negative breast cancer), melanoma, liver cancer, bladder cancer, stomach cancer, esophageal cancer, and chronic myelogenous leukemia. In particular embodiments, the cancer is selected from the group consisting of head and neck cancer, non-small cell lung cancer, esophageal cancer, gastric cancer, hepatic cancer, pancreatic cancer, colorectal cancer, breast cancer, endometrial cancer, ovarian cancer, soft-tissue sarcoma, bladder cancer, prostate cancer, renal cancer, and melanoma.

In certain embodiments, the cancer is selected from adenocarcinoma, bladder cancer, bowel cancer, breast cancer (e.g., triple negative breast cancer), cervical cancer, colorectal cancer, esophageal cancer, gastric cancer, head and neck cancer (e.g., head and neck squamous cell carcinoma), lung cancer (e.g., non-small cell lung cancer), mesothelioma, ovarian cancer, pancreatic cancer, prostate cancer, renal cancer, squamous cell carcinoma, uterine cancer, retinoblastoma, and Ewing sarcoma.

The cancer may be, e.g., at an early, intermediate, late, locally advanced, or metastatic stage, and may be relapsed or refractory to other therapeutics, or there may be no standard therapy available.

“Treat,” “treating,” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment. In some embodiments, therapeutic use of an antibody or antigen-binding portion thereof described herein will result in delayed tumor growth, elimination of cancer cells, tumor shrinkage/regression, increased survival, slowed or decreased metastasis, or other clinical endpoints desired by healthcare professionals.

The 5T4-binding proteins of the present disclosure may be administered without additional therapeutic treatments, i.e., as a stand-alone therapy (monotherapy). Alternatively, treatment with the 5T4-binding proteins may include at least one additional therapeutic treatment (combination therapy), e.g., an anti-cancer agent (such as a chemotherapeutic agent, an anti-neoplastic agent, or an anti-angiogenic agent), a vaccine (such as a tumor vaccine), a targeted agent (e.g., a kinase inhibitor), an immunotherapeutic agent (e.g., a checkpoint inhibitor), hormone therapy, radiation therapy, etc.

The 5T4-binding proteins also are useful in diagnostic processes (e.g., in vitro or ex vivo). For example, the 5T4-binding proteins can be used to detect and/or measure the level of 5T4 in a biological sample from a patient (e.g., a tumor biopsy, a tissue sample, or a blood sample). Suitable detection and measurement methods include immunological methods such as flow cytometry, enzyme-linked immunosorbent assays (ELISA), chemiluminescence assays, radioimmunoassays, and immunohistochemistry. The present disclosure further encompasses kits (e.g., diagnostic kits) comprising the antibodies, antigen-binding portions, or binding proteins described herein.

The pharmaceutical composition may be administered to a patient (e.g., a human) in need thereof in a therapeutically effective amount. A “therapeutically effective amount” is an amount that will relieve to some extent one or more of the symptoms of the disease being treated. A therapeutically effective amount of an anti-cancer therapeutic may, for example, result in delayed cancer growth; elimination of cancer cells; tumor shrinkage; increased survival; prevented, decreased, or slowed metastasis; or other clinical endpoints desired by healthcare professionals.

The pharmaceutical compositions herein may be delivered to the patient through parenteral administration, e.g., selected from subcutaneous, intraperitoneal, intramuscular, intrasternal, intracisternal, intravenous, intraarterial, intrathecal, intraurethral, intracranial, intratumoral, and intrasynovial injection or infusions. Particular embodiments include the intravenous route (e.g., intravenous infusion) and the subcutaneous route (e.g., subcutaneous injection).

Pharmaceutical compositions of the present invention and methods for their preparation will be readily apparent to those skilled in the art. Such compositions and methods for their preparation may be found, for example, in Remington's Pharmaceutical Sciences, 19^{th} Edition (Mack Publishing Company, 1995). Pharmaceutical compositions are preferably manufactured under GMP (good manufacturing practices) conditions.

A pharmaceutical composition of the present disclosure may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Formulations of a pharmaceutical composition suitable for parenteral administration typically comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

As used herein, the percent identity of two amino acid sequences (or of two nucleic acid sequences) may be obtained by, e.g., BLAST® using default parameters (available at the U.S. National Library of Medicine's National Center for Biotechnology Information website). In some embodiments, the length of a query sequence aligned for comparison purposes is at least 30% (e.g., at least 40, 50, 60, 70, 80, or 90%) of the length of the reference sequence.

According to the present disclosure, back-references in the dependent claims are meant as short-hand writing for a direct and unambiguous disclosure of each and every combination of claims that is indicated by the back-reference. Any compound disclosed herein can be used in any of the treatment methods disclosed herein, wherein the individual to be treated is as defined anywhere herein.

In order that this invention may be better understood, the following examples are set forth. These examples are for purposes of illustration only and are not to be construed as limiting the scope of the invention in any manner.

### EXAMPLES

### Example 1: Antibody Discovery Campaign 1

### Antibody Generation and Isolation

Alloy ATX-GK mice (n=5 mice per immunization cohort), which produce antibodies with fully human V_{H} and V_{L}, were immunized with recombinant human 5T4 extra-cellular domain (ECD) Fc protein (immunization cohort 1) or with recombinant human 5T4 ECD Avi-, his-tagged protein (immunization cohort 2). A repetitive immunization at multiple sites (RIMMS) protocol was used for immunization. Following immunization, B cells were collected from spleen or lymph nodes and single 5T4-reactive memory B cells (>1,500) were isolated by flow cytometry using biotinylated cynomolgus 5T4 ECD Avi-, his-tagged protein complexed with neutravidin-Dylight™ 650. Antibody genes from single cells were amplified by RT-PCR and sequenced. Three control antibodies (SLX-2001, SLX-2002 and SLX-2003) and approximately 100 unique antibody sequences were expressed as human IgG₁ in CHO cells. Antibodies were purified from culture supernatant using protein A and was further characterized, as described below.

### Example 2: Antibody Characterization

Antibody purity was assessed by non-reducing CE-SDS using the LabChip® GXII Touch™ system. As summarized in Table 1, the majority of the antibodies (73 of 87 tested) displayed purity >9000. Antibodies SLX-2001, SLX-2002, and SLX-2003 are control antibodies. In addition, the monomeric content of the antibodies was assessed by analytical SEC. The majority of antibodies displayed high monomeric content (>950%), as summarized in Table 1.

TABLE 1
Antibody Purity and Monomeric Content

| | | | Monomeric |
|---|---|---|---|
| | Epitope | Purity | Content |
| Antibody | Bin | (%) | (%) |
| SLX-2001 | 2 | >95 | >95 |
| SLX-2002 | 1b | >95 | >95 |
| SLX-2003 | 1a | >95 | >95 |
| SLX-2016 | 2 | 60.9 | >95 |
| SLX-2017 | 1d | >95 | >95 |
| SLX-2018 | 1d | >95 | >95 |
| SLX-2019 | 4e | 56.7 | >95 |
| SLX-2020 | 4e | >95 | >95 |
| SLX-2021 | 5a | >95 | >95 |
| SLX-2022 | 3a | >95 | >95 |
| SLX-2023 | 2 | 79.0 | >95 |
| SLX-2024 | unknown | >95 | >95 |
| SLX-2025 | 3a | 93.2 | >95 |
| SLX-2026 | 3a | 57.4 | >95 |
| SLX-2027 | 3a | >95 | >95 |
| SLX-2028 | 2 | >95 | >95 |
| SLX-2029 | 7a | >95 | >95 |
| SLX-2030 | 1d | >95 | >95 |
| SLX-2031 | 3a | >95 | >95 |
| SLX-2032 | 2 | >95 | >95 |
| SLX-2033 | 1d | >95 | >95 |
| SLX-2034 | 2 | >95 | >95 |
| SLX-2035 | 2 | >95 | >95 |
| SLX-2036 | 1d | 92.5 | >95 |
| SLX-2037 | 2 | >95 | >95 |
| SLX-2038 | unknown | >95 | >95 |
| SLX-2039 | unknown | ND | 89.9 |
| SLX-2040 | 4c | >95 | >95 |
| SLX-2041 | 3a | >95 | >95 |
| SLX-2042 | 4a | >95 | >95 |
| SLX-2043 | 4d | >95 | >95 |
| SLX-2044 | 1d | >95 | >95 |
| SLX-2045 | 1d | 58.5 | >95 |
| SLX-2046 | 1d | >95 | >95 |
| SLX-2047 | 1d | >95 | >95 |
| SLX-2048 | 2 | 88.1 | >95 |
| SLX-2049 | unknown | 68.1 | >95 |
| SLX-2050 | 1d | >95 | >95 |
| SLX-2051 | 4e | >95 | >95 |
| SLX-2052 | 3a | >95 | >95 |
| SLX-2053 | 4f | 89.2 | >95 |
| SLX-2054 | 4b | >95 | >95 |
| SLX-2055 | 4b | 72.1 | >95 |
| SLX-2056 | unknown | >95 | >95 |
| SLX-2057 | 4b | >95 | >95 |
| SLX-2058 | 3a | >95 | >95 |
| SLX-2059 | 1d | >95 | >95 |
| SLX-2060 | 4b | >95 | >95 |
| SLX-2061 | 4b | >95 | >95 |
| SLX-2062 | 2 | >95 | >95 |
| SLX-2063 | 1f | >95 | >95 |
| SLX-2064 | 1d | >95 | >95 |
| SLX-2065 | 3b | 88.5 | >95 |
| SLX-2066 | 3b | >95 | >95 |
| SLX-2067 | unknown | >95 | >95 |
| SLX-2068 | unknown | >95 | >95 |
| SLX-2069 | 6a | >95 | >95 |
| SLX-2070 | 1e | >95 | >95 |
| SLX-2071 | 2 | >95 | >95 |
| SLX-2072 | 1d | >95 | >95 |
| SLX-2073 | 2 | >95 | >95 |
| SLX-2074 | unknown | >95 | >95 |
| SLX-2075 | 3c | >95 | >95 |
| SLX-2076 | unknown | >95 | >95 |
| SLX-2077 | 3a | >95 | >95 |
| SLX-2078 | unknown | >95 | >95 |
| SLX-2079 | 3a | >95 | >95 |
| SLX-2080 | 1c | >95 | >95 |
| SLX-2081 | 3a | >95 | >95 |
| SLX-2082 | 2 | >95 | >95 |
| SLX-2083 | 3a | >95 | >95 |
| SLX-2084 | 2 | >95 | >95 |
| SLX-2085 | 3a | >95 | >95 |
| SLX-2086 | 4e | >95 | >95 |
| SLX-2087 | 2 | >95 | >95 |
| SLX-2088 | 1d | 90.5 | >95 |
| SLX-2089 | 4b | >95 | 94.6 |
| SLX-2090 | 1c | >95 | >95 |
| SLX-2091 | 2 | >95 | >95 |
| SLX-2092 | 4b | >95 | >95 |
| SLX-2093 | 2 | >95 | >95 |
| SLX-2094 | 2 | >95 | >95 |
| SLX-2095 | 3c | 85.0 | >95 |
| SLX-2096 | 2 | >95 | >95 |
| SLX-2097 | 2 | >95 | >95 |
| SLX-2098 | 2 | >95 | >95 |
| SLX-2099 | 2 | >95 | >95 |
| SLX-2100 | 2 | >95 | >95 |
| SLX-2101 | 2 | >95 | >95 |
| SLX-2102 | 2 | >95 | >95 |
| SLX-2103 | 2 | >95 | >95 |

ND: not determined

Next, the reactivity of the antibodies with recombinant human, cynomolgus, and mouse 5T4 protein was characterized using a Carterra LSA instrument. Antibodies immobilized on the chip were probed with human, cynomolgus and mouse Fc-tagged 5T4 ECD protein and the binding was expressed as Response Units (RU), normalized for the amount of antibody captured. Because the mice were immunized with human 5T4 ECD protein and the B cells were isolated with cynomolgus 5T4 ECD protein, it was expected that the antibodies would be reactive with both species. In fact, all antibodies tested were cross-reactive with both species (Table 2). In addition, some of the antibodies (23/88) also recognized mouse 5T4 ECD (relative RU >50, Table 2).

TABLE 2
Binding and Kinetic Data

| Antibody | Human | Cynomolgus | Mouse |
|---|---|---|---|
| SLX-2016 | 613 | 582 | −10 |
| SLX-2017 | 683 | 696 | −3 |
| SLX-2018 | 845 | 895 | 1 |
| SLX-2019 | 640 | 672 | 408 |
| SLX-2020 | 447 | 474 | 154 |
| SLX-2021 | 728 | 641 | 0 |
| SLX-2022 | 619 | 676 | 2 |
| SLX-2023 | 492 | 584 | 12 |
| SLX-2024 | 485 | 73 | −13 |
| SLX-2025 | 836 | 861 | −3 |
| SLX-2026 | 631 | 649 | 23 |
| SLX-2027 | 718 | 727 | 3 |
| SLX-2028 | 534 | 523 | 32 |
| SLX-2029 | 505 | 529 | 13 |
| SLX-2030 | 713 | 691 | 3 |
| SLX-2031 | 783 | 780 | −6 |
| SLX-2032 | 518 | 570 | 379 |
| SLX-2033 | 890 | 916 | −2 |
| SLX-2034 | 682 | 697 | 736 |
| SLX-2035 | 687 | 685 | 91 |
| SLX-2036 | 684 | 623 | 16 |
| SLX-2037 | 563 | 573 | 603 |
| SLX-2038 | 692 | 621 | 4 |
| SLX-2039 | 653 | 521 | 6 |
| SLX-2040 | 717 | 763 | 35 |
| SLX-2041 | 702 | 649 | 21 |
| SLX-2042 | 539 | 570 | 57 |
| SLX-2043 | 527 | 563 | −11 |
| SLX-2044 | 744 | 783 | −3 |
| SLX-2045 | 850 | 878 | 17 |
| SLX-2046 | 819 | 817 | 0 |
| SLX-2047 | 765 | 736 | 2 |
| SLX-2048 | 655 | 686 | −4 |
| SLX-2049 | 561 | 168 | −6 |
| SLX-2050 | 828 | 794 | 12 |
| SLX-2051 | 791 | 818 | 19 |
| SLX-2052 | 750 | 746 | −4 |
| SLX-2053 | 595 | 636 | 158 |
| SLX-2054 | 644 | 693 | 304 |
| SLX-2055 | 663 | 677 | 368 |
| SLX-2056 | 555 | 219 | 17 |
| SLX-2057 | 552 | 572 | 487 |
| SLX-2058 | 794 | 813 | 3 |
| SLX-2059 | 862 | 871 | 13 |
| SLX-2060 | 676 | 740 | 104 |
| SLX-2061 | 651 | 681 | 51 |
| SLX-2062 | 633 | 688 | −2 |
| SLX-2063 | 824 | 519 | 7 |
| SLX-2064 | 984 | 961 | 16 |
| SLX-2065 | 752 | 766 | −8 |
| SLX-2066 | 775 | 778 | −5 |
| SLX-2067 | 486 | 133 | −3 |
| SLX-2068 | 548 | 321 | 10 |
| SLX-2069 | 814 | 609 | −2 |
| SLX-2070 | 721 | 718 | −5 |
| SLX-2071 | 696 | 733 | 466 |
| SLX-2072 | 838 | 647 | −3 |
| SLX-2073 | 618 | 619 | 607 |
| SLX-2074 | 486 | 156 | 7 |
| SLX-2075 | 621 | 479 | 7 |
| SLX-2076 | 707 | 550 | 2 |
| SLX-2077 | 829 | 851 | −3 |
| SLX-2078 | 704 | 319 | −10 |
| SLX-2079 | 667 | 610 | −5 |
| SLX-2080 | 818 | 836 | 2 |
| SLX-2081 | 564 | 451 | −5 |
| SLX-2082 | 518 | 536 | 10 |
| SLX-2083 | 512 | 452 | 1 |
| SLX-2084 | 539 | 564 | 10 |
| SLX-2085 | 459 | 389 | −1 |
| SLX-2086 | 519 | 548 | 113 |
| SLX-2087 | 564 | 579 | 28 |
| SLX-2088 | 779 | 786 | −11 |
| SLX-2089 | 699 | 715 | 654 |
| SLX-2090 | 716 | 567 | 9 |
| SLX-2091 | 416 | 353 | −2 |
| SLX-2092 | 653 | 673 | 539 |
| SLX-2093 | 714 | 729 | 222 |
| SLX-2094 | 694 | 717 | 778 |
| SLX-2095 | 579 | 338 | 5 |
| SLX-2096 | 519 | 534 | −10 |
| SLX-2097 | 688 | 711 | 499 |
| SLX-2098 | 605 | 604 | 356 |
| SLX-2099 | 496 | 496 | 354 |
| SLX-2100 | 468 | 507 | −7 |
| SLX-2101 | 446 | 491 | −1 |
| SLX-2102 | 498 | 578 | 0 |
| SLX-2103 | 562 | 616 | −3 |

The kinetics of the antibodies' binding to recombinant human and cynomolgus 5T4 Avi-, His-tagged protein was characterized using a Carterra LSA instrument. Multiple antibodies displaying very high affinity (<1 nM) binding to human 5T4 were identified (SLX-2034, SLX-2035, SLX-2059, SLX-2065, SLX-2073, SLX-2093, and SLX-2094), as summarized in Table 3. Likewise, multiple antibodies displaying very high affinity (<1 nM) binding to cynomolgus 5T4 were identified (SLX-2017, SLX-2018, SLX-2064, SLX-2065, SLX-2066, SLX-2073, SLX-2080, SLX-2093).

TABLE 3
Antibody Kinetics for Binding to Human and Cynomolgus 5T4

| | Human 5T4 Avi-, His-tag | | | Cynomolgus 5T4 Avi-, His-tag | | |
|---|---|---|---|---|---|---|
| Antibody | ka (1/Ms) | kd (1/s) | K_{D} (M) | ka (1/Ms) | kd (1/s) | K_{D} (M) |
| SLX-2001 | 1.1E+05 | 2.6E−03 | 2.4E−08 | 2.2E+05 | 8.9E−04 | 4.1E−09 |
| SLX-2002 | 8.6E+03 | 2.3E−04 | 2.7E−08 | 7.0E+05 | 1.0E−05 | 1.4E−11 |
| SLX-2003 | 1.5E+05 | 8.2E−04 | 5.5E−09 | 3.6E+05 | 8.3E−04 | 2.3E−09 |
| SLX-2016 | 1.9E+04 | 1.0E−03 | 5.3E−08 | 3.3E+04 | 2.1E−03 | 6.2E−08 |
| SLX-2017 | 2.5E+05 | 7.0E−04 | 2.8E−09 | 6.6E+05 | 3.9E−04 | 5.9E−10 |
| SLX-2018 | 4.1E+05 | 4.5E−03 | 1.1E−08 | 1.5E+06 | 1.2E−03 | 8.0E−10 |
| SLX-2019 | 1.9E+05 | 2.7E−02 | 1.5E−07 | 2.5E+05 | 5.0E−02 | 2.0E−07 |
| SLX-2020 | 1.2E+05 | 9.3E−03 | 7.8E−08 | 1.3E+05 | 2.2E−02 | 1.7E−07 |
| SLX-2021 | 1.2E+04 | 2.4E−03 | 1.9E−07 | 8.1E+05 | 2.4E−03 | 3.0E−09 |
| SLX-2022 | 9.4E+05 | 5.2E−01 | 5.5E−07 | 2.2E+06 | 1.8E+00 | 8.3E−07 |
| SLX-2023 | 2.8E+04 | 6.4E−05 | 2.3E−09 | 1.7E+04 | 4.9E−04 | 3.0E−08 |
| SLX-2024 | N.B. | N.B. | N.B. | 1.9E+04 | 3.7E−02 | 1.9E−06 |
| SLX-2025 | 4.6E+05 | 9.7E−03 | 2.1E−08 | 9.4E+05 | 4.3E−03 | 4.6E−09 |
| SLX-2026 | 1.9E+04 | 2.6E−03 | 1.4E−07 | 5.1E+04 | 5.3E−04 | 1.0E−08 |
| SLX-2027 | 3.7E+04 | 2.7E−03 | 7.2E−08 | 7.8E+04 | 2.4E−04 | 3.0E−09 |
| SLX-2028 | 6.1E+03 | 5.9E−04 | 9.8E−08 | 3.6E+04 | 6.3E−03 | 1.7E−07 |
| SLX-2029 | 1.5E+05 | 9.7E−02 | 6.7E−07 | 4.4E+05 | 1.3E−01 | 2.9E−07 |
| SLX-2030 | 2.1E+05 | 2.9E−03 | 1.3E−08 | 9.0E+05 | 9.9E−04 | 1.1E−09 |
| SLX-2031 | 3.9E+05 | 1.3E−02 | 3.3E−08 | 8.1E+05 | 1.1E−02 | 1.3E−08 |
| SLX-2032 | 6.6E+04 | 4.2E−03 | 6.3E−08 | 9.7E+04 | 1.7E−02 | 1.8E−07 |
| SLX-2033 | 5.0E+05 | 6.3E−03 | 1.3E−08 | 1.1E+06 | 2.1E−03 | 1.9E−09 |
| SLX-2034 | 2.2E+05 | 1.5E−04 | 6.9E−10 | 2.6E+05 | 4.5E−04 | 1.8E−09 |
| SLX-2035 | 8.7E+04 | 6.3E−05 | 7.3E−10 | 2.1E+05 | 1.0E−05 | 4.9E−11 |
| SLX-2036 | 1.1E+05 | 4.8E−03 | 4.3E−08 | 5.3E+05 | 5.6E−03 | 1.1E−08 |
| SLX-2037 | 1.3E+05 | 2.4E−04 | 1.9E−09 | 2.0E+05 | 5.8E−04 | 2.8E−09 |
| SLX-2038 | P.F | P.F. | P.F. | 1.2E+06 | 1.1E−03 | 9.1E−10 |
| SLX-2039 | P.F. | P.F. | P.F. | 1.8E+05 | 5.9E−04 | 3.3E−09 |
| SLX-2040 | P.F./W.B. | P.F./W.B. | P.F./W.B. | 1.9E+05 | 2.8E−02 | 1.5E−07 |
| SLX-2041 | P.F./W.B. | P.F./W.B. | P.F./W.B. | 5.1E+04 | 6.5E−04 | 1.3E−08 |
| SLX-2042 | 5.2E+04 | 9.7E−03 | 1.9E−07 | 4.5E+04 | 1.8E−02 | 4.0E−07 |
| SLX-2043 | P.F./W.B. | P.F./W.B. | P.F./W.B. | 1.1E+05 | 3.3E−02 | 2.9E−07 |
| SLX-2044 | 2.5E+05 | 1.0E−03 | 4.0E−09 | 9.5E+05 | 9.7E−04 | 1.0E−09 |
| SLX-2045 | 4.2E+05 | 1.6E−03 | 3.8E−09 | 1.2E+06 | 5.8E−04 | 4.7E−10 |
| SLX-2046 | 1.7E+05 | 5.1E−03 | 3.0E−08 | 4.9E+05 | 7.0E−03 | 1.4E−08 |
| SLX-2047 | 2.1E+05 | 2.3E−02 | 1.1E−07 | 6.3E+05 | 1.6E−02 | 2.5E−08 |
| SLX-2048 | 5.6E+04 | 1.6E−04 | 2.9E−09 | 8.3E+04 | 1.7E−04 | 2.0E−09 |
| SLX-2049 | N.B. | N.B. | N.B. | 1.8E+04 | 6.4E−03 | 3.6E−07 |
| SLX-2050 | 2.8E+05 | 1.1E−02 | 3.7E−08 | 1.2E+06 | 6.2E−03 | 5.1E−09 |
| SLX-2051 | 2.2E+05 | 3.4E−02 | 1.5E−07 | 2.1E+05 | 2.9E−02 | 1.4E−07 |
| SLX-2052 | 4.4E+05 | 2.3E−03 | 5.1E−09 | 6.6E+05 | 1.8E−03 | 2.7E−09 |
| SLX-2053 | 3.5E+04 | 9.8E−03 | 2.8E−07 | 6.1E+04 | 3.2E−02 | 5.3E−07 |
| SLX-2054 | 1.3E+05 | 2.3E−03 | 1.8E−08 | 1.4E+05 | 1.9E−03 | 1.3E−08 |
| SLX-2055 | 6.9E+04 | 9.1E−03 | 1.3E−07 | 7.2E+04 | 1.2E−02 | 1.6E−07 |
| SLX-2056 | P.F./W.B. | P.F./W.B. | P.F./W.B. | 2.3E+04 | 9.1E−03 | 4.0E−07 |
| SLX-2057 | 1.9E+05 | 1.3E−03 | 7.2E−09 | 1.2E+05 | 2.4E−03 | 1.9E−08 |
| SLX-2058 | 3.8E+04 | 1.2E−03 | 3.1E−08 | 9.7E+04 | 8.6E−05 | 8.9E−10 |
| SLX-2059 | 3.4E+05 | 6.9E−05 | 2.1E−10 | 8.3E+05 | 1.4E−04 | 1.7E−10 |
| SLX-2060 | 4.0E+04 | 4.7E−03 | 1.2E−07 | 1.1E+05 | 2.9E−02 | 2.7E−07 |
| SLX-2061 | 3.0E+05 | 7.0E−02 | 2.3E−07 | 3.6E+05 | 9.3E−02 | 2.6E−07 |
| SLX-2062 | 2.2E+04 | 6.8E−04 | 3.1E−08 | 3.7E+04 | 7.0E−05 | 1.9E−09 |
| SLX-2063 | 4.6E+03 | 2.0E−03 | 4.4E−07 | 2.6E+05 | 1.7E−03 | 6.7E−09 |
| SLX-2064 | 1.8E+05 | 1.2E−03 | 6.7E−09 | 5.6E+05 | 4.8E−04 | 8.6E−10 |
| SLX-2065 | 1.9E+05 | 1.2E−04 | 6.1E−10 | 2.2E+05 | 1.2E−04 | 5.5E−10 |
| SLX-2066 | 1.2E+05 | 1.4E−04 | 1.1E−09 | 1.8E+05 | 8.5E−05 | 4.7E−10 |
| SLX-2067 | N.B. | N.B. | N.B. | 2.4E+04 | 4.7E−02 | 1.9E−06 |
| SLX-2068 | W.B. | W.B. | W.B. | 3.2E+05 | 1.1E−01 | 3.3E−07 |
| SLX-2069 | 5.1E+03 | 1.9E−03 | 3.8E−07 | 3.4E+05 | 1.4E−03 | 4.1E−09 |
| SLX-2070 | 2.5E+05 | 1.7E−03 | 6.7E−09 | 7.7E+05 | 2.4E−03 | 3.0E−09 |
| SLX-2071 | 6.9E+04 | 2.2E−04 | 3.1E−09 | 1.4E+05 | 4.6E−04 | 3.2E−09 |
| SLX-2072 | 7.4E+03 | 2.7E−03 | 3.7E−07 | 5.9E+05 | 4.7E−03 | 7.9E−09 |
| SLX-2073 | 1.8E+05 | 1.0E−05 | 5.4E−11 | 3.0E+05 | 3.9E−05 | 1.3E−10 |
| SLX-2074 | N.B. | N.B. | N.B. | 7.9E+04 | 4.6E−02 | 5.8E−07 |
| SLX-2075 | 5.9E+03 | 2.3E−03 | 4.0E−07 | 2.7E+05 | 1.3E−03 | 4.8E−09 |
| SLX-2076 | N.B. | N.B. | N.B. | 2.6E+06 | 8.9E−01 | 3.4E−07 |
| SLX-2077 | 3.3E+05 | 8.2E−03 | 2.5E−08 | 5.3E+05 | 2.5E−03 | 4.7E−09 |
| SLX-2078 | N.B. | N.B. | N.B. | 5.8E+04 | 3.2E−02 | 5.5E−07 |
| SLX-2079 | 3.6E+04 | 2.0E−02 | 5.4E−07 | 5.7E+04 | 1.2E−03 | 2.1E−08 |
| SLX-2080 | 1.5E+05 | 9.6E−04 | 6.4E−09 | 6.0E+05 | 3.6E−04 | 5.9E−10 |
| SLX-2081 | 6.0E+03 | 7.4E−04 | 1.2E−07 | 1.1E+04 | 2.2E−04 | 2.0E−08 |
| SLX-2082 | 3.8E+04 | 2.0E−03 | 5.3E−08 | 8.9E+04 | 1.6E−04 | 1.8E−09 |
| SLX-2083 | 1.0E+04 | 3.4E−03 | 3.4E−07 | 2.1E+04 | 2.1E−03 | 1.0E−07 |
| SLX-2084 | 4.8E+04 | 5.0E−04 | 1.0E−08 | 5.7E+04 | 1.2E−04 | 2.0E−09 |
| SLX-2085 | 3.7E+03 | 2.3E−04 | 6.3E−08 | 1.2E+04 | 1.3E−04 | 1.1E−08 |
| SLX-2086 | 4.0E+04 | 4.7E−03 | 1.2E−07 | 3.4E+04 | 1.1E−02 | 3.2E−07 |
| SLX-2087 | 4.7E+04 | 4.2E−04 | 9.0E−09 | 5.5E+04 | 1.3E−04 | 2.4E−09 |
| SLX-2088 | 4.3E+05 | 3.6E−03 | 8.2E−09 | 1.1E+06 | 1.1E−03 | 1.0E−09 |
| SLX-2089 | 2.0E+05 | 5.6E−04 | 2.7E−09 | 3.2E+05 | 4.6E−04 | 1.5E−09 |
| SLX-2090 | 2.4E+05 | 1.7E−01 | 6.8E−07 | 3.8E+05 | 1.7E−02 | 4.5E−08 |
| SLX-2091 | 7.4E+03 | 8.3E−05 | 1.1E−08 | 1.5E+04 | 2.3E−04 | 1.6E−08 |
| SLX-2092 | 1.6E+05 | 1.3E−03 | 7.9E−09 | 1.9E+05 | 6.6E−04 | 3.4E−09 |
| SLX-2093 | 8.7E+04 | 3.4E−05 | 3.9E−10 | 1.9E+05 | 1.0E−05 | 5.1E−11 |
| SLX-2094 | 2.6E+05 | 1.0E−04 | 3.9E−10 | 1.9E+05 | 2.0E−04 | 1.1E−09 |
| SLX-2095 | P.F. | P.F. | P.F. | 8.0E+04 | 2.7E−03 | 3.4E−08 |
| SLX-2096 | 3.3E+04 | 3.2E−03 | 9.6E−08 | 2.9E+04 | 8.1E−03 | 2.8E−07 |
| SLX-2097 | 1.3E+05 | 3.6E−03 | 2.8E−08 | 1.0E+05 | 1.2E−02 | 1.2E−07 |
| SLX-2098 | 7.7E+04 | 1.7E−03 | 2.3E−08 | 9.8E+04 | 6.4E−03 | 6.5E−08 |
| SLX-2099 | 1.6E+04 | 2.3E−04 | 1.4E−08 | 2.2E+04 | 1.3E−03 | 6.0E−08 |
| SLX-2100 | 2.1E+04 | 4.2E−03 | 2.0E−07 | 1.9E+04 | 8.3E−03 | 4.4E−07 |
| SLX-2101 | 2.6E+04 | 1.8E−03 | 6.8E−08 | 3.2E+04 | 3.1E−03 | 9.5E−08 |
| SLX-2102 | 1.4E+04 | 7.5E−03 | 5.5E−07 | 1.9E+04 | 1.4E−02 | 7.6E−07 |
| SLX-2103 | 3.1E+04 | 2.7E−03 | 8.6E−08 | 2.7E+04 | 5.0E−03 | 1.9E−07 |

P.F.: poor fit.
W.B.: weak binding.
N.B.: no binding

Antibody binding to human 5T4-expressing tumor cells (MCF7) and non-expressing tumor cells (Jurkat) was assessed. Full titrations on MCF7 cells were performed to obtain EC₅₀ values for tumor cell binding. All antibodies were tested for non-selective cell binding by measuring the MFI for binding to Jurkat cells at 100 nM. These values were compared to the MFI for binding to MCF7 cells at 100 nM.

Most antibodies (80/88) bound MCF7 cells with an MFI >1,000 at 100 nM (Table 4). The cell binding was generally strong with many clones binding with EC₅₀<1 nM. Furthermore, the majority of the cell binding antibodies were selective for binding to MCF7 compared to Jurkat cells. Specifically, only 7/80 antibodies that bound MCF7 cells with an MFI >1,000 displayed Jurkat binding above background (MFI >30). No direct correlation between binding affinity to soluble 5T4 (Table 3) and binding EC₅₀ to MCF7 cells (Table 4) was observed.

TABLE 4
Antibody Binding to 5T4-expressing (MCF7)
and Non-Expressing (Jurkat) Tumor Cells

| | MCF7 EC₅₀ | MCF7 MFI | Jurkat MFI |
|---|---|---|---|
| Antibody | (nM) | (100 nM) | (100 nM) |
| SLX-2001 | N.D. | N.D. | N.D. |
| SLX-2002 | 0.52 | 10895 | 37.2 |
| SLX-2003 | 0.51 | 10846 | 29.5 |
| SLX-2016 | N.P. | 4032 | 19.3 |
| SLX-2017 | 0.49 | 11095 | 24.4 |
| SLX-2018 | 0.29 | 10179 | 29.5 |
| SLX-2019 | W.B. | 380 | 23.1 |
| SLX-2020 | N.P. | 3101 | 20.5 |
| SLX-2021 | 0.59 | 10318 | 20.5 |
| SLX-2022 | N.P. | 3910 | 25.7 |
| SLX-2023 | W.B. | 1373 | 20.5 |
| SLX-2024 | W.B. | 676 | 18 |
| SLX-2025 | 0.97 | 10364 | 20.5 |
| SLX-2026 | N.P. | 11070 | 23.1 |
| SLX-2027 | N.P. | 11020 | 25.7 |
| SLX-2028 | W.B. | 2099 | 20.5 |
| SLX-2029 | W.B. | 143 | 18 |
| SLX-2030 | 0.61 | 10772 | 23.1 |
| SLX-2031 | 0.42 | 10110 | 24.4 |
| SLX-2032 | W.B. | 540 | 19.3 |
| SLX-2033 | 0.35 | 10920 | 20.5 |
| SLX-2034 | N.P. | 7349 | 23.1 |
| SLX-2035 | N.P. | 7142 | 29.5 |
| SLX-2036 | 2.03 | 11507 | 29.5 |
| SLX-2037 | W.B. | 2837 | 19.3 |
| SLX-2038 | 0.76 | 11507 | 23.1 |
| SLX-2039 | 4.22 | 11507 | 86.1 |
| SLX-2040 | W.B. | 1884 | 18 |
| SLX-2041 | N.P. | 11377 | 23.1 |
| SLX-2042 | W.B. | 2593 | 18 |
| SLX-2043 | W.B. | 941 | 18 |
| SLX-2044 | 0.79 | 11197 | 23.1 |
| SLX-2045 | 0.18 | 11171 | 20.5 |
| SLX-2046 | 0.73 | 11045 | 21.8 |
| SLX-2047 | 0.36 | 10530 | 50.1 |
| SLX-2048 | N.P. | 7680 | 20.5 |
| SLX-2049 | N.P. | 5268 | 18 |
| SLX-2050 | 0.86 | 11045 | 23.1 |
| SLX-2051 | N.P. | 5427 | 18 |
| SLX-2052 | 0.52 | 10650 | 25.7 |
| SLX-2053 | W.B. | 463 | 18 |
| SLX-2054 | N.P. | 9074 | 19.3 |
| SLX-2055 | N.P. | 5544 | 18 |
| SLX-2056 | N.P. | 6616 | 20.5 |
| SLX-2057 | N.P. | 8933 | 18 |
| SLX-2058 | N.P. | 14184 | 23.1 |
| SLX-2059 | 0.40 | 12381 | 24.4 |
| SLX-2060 | N.P. | 2275 | 18 |
| SLX-2061 | W.B. | 4041 | 28.2 |
| SLX-2062 | W.B. | 1867 | 20.5 |
| SLX-2063 | 1.53 | 9427 | 28.2 |
| SLX-2064 | 0.58 | 11963 | 20.5 |
| SLX-2065 | 1.00 | 11746 | 32.1 |
| SLX-2066 | 1.17 | 11990 | 29.5 |
| SLX-2067 | W.B. | 1006 | 18 |
| SLX-2068 | W.B. | 1251 | 18 |
| SLX-2069 | 1.30 | 11481 | 21.8 |
| SLX-2070 | 0.25 | 11773 | 20.5 |
| SLX-2071 | 9.98 | 10920 | 23.1 |
| SLX-2072 | 0.43 | 11377 | 23.1 |
| SLX-2073 | N.P. | 11586 | 35.9 |
| SLX-2074 | W.B. | 1881 | 27 |
| SLX-2075 | 4.94 | 11936 | 48.8 |
| SLX-2076 | N.P. | 3568 | 20.5 |
| SLX-2077 | 0.56 | 11800 | 24.4 |
| SLX-2078 | N.P. | 6645 | 19.3 |
| SLX-2079 | 17.71 | 11534 | 27 |
| SLX-2080 | 0.50 | 12381 | 24.4 |
| SLX-2081 | N.P. | 8913 | 25.7 |
| SLX-2082 | N.P. | 8208 | 20.5 |
| SLX-2083 | N.P. | 11248 | 24.4 |
| SLX-2084 | N.P. | 8355 | 20.5 |
| SLX-2085 | N.P. | 10506 | 23.1 |
| SLX-2086 | N.P. | 3266 | 37.2 |
| SLX-2087 | N.P. | 8581 | 21.8 |
| SLX-2088 | 0.20 | 11827 | 20.5 |
| SLX-2089 | 20.41 | 10846 | 23.1 |
| SLX-2090 | 0.18 | 10578 | 20.5 |
| SLX-2091 | N.P. | 3221 | 20.5 |
| SLX-2092 | N.P. | 9795 | 25.7 |
| SLX-2093 | N.P. | 7869 | 23.1 |
| SLX-2094 | N.P. | 3934 | 20.5 |
| SLX-2095 | 5.02 | 9906 | 20.5 |
| SLX-2096 | W.B. | 580 | 18 |
| SLX-2097 | N.P. | 6588 | 19.3 |
| SLX-2098 | N.P. | 6987 | 18 |
| SLX-2099 | N.P. | 3339 | 18 |
| SLX-2100 | W.B. | 2244 | 23.1 |
| SLX-2101 | W.B. | 2227 | 38.5 |
| SLX-2102 | W.B. | 464 | 18 |
| SLX-2103 | W.B. | 2283 | 18 |

N.D.: not determined;
N.P.: no plateau achieved,
W.B.: weak binding

The antibodies were characterized by epitope binning using a Carterra LSA instrument and standard protocols (immobilization of candidate antibodies, followed by injection of 5T4 antigen, followed by injection of candidate antibodies). The cross-competition data were analyzed using the Carterra's Epitope™ Software (epitope binning analysis software). The combined dendograms created based on the cross-competition data were utilized to develop community plots. Epitope binning identified four dominant communities (1-4) and multiple closely-related sub-groups (Community 1 consists of sub-groups 1a-1f, Community 2 is a single group, Community 3 consists of sub-groups 3a-3c, and Community 4 consists of sub-groups 4a-4f). Three antibodies did not belong directly within communities 1-4 and were designated as epitope bins 5, 6, and 7. The epitope bins of the three control antibodies were also determined, with SLX-2002 and SLX-2003 belonging to sub-groups 1b and 1a, respectively and SLX-2001 belonging to epitope bin 2. The epitope bin designation for all the antibodies is provided in Table 1.

### Example 3: Removal of Potential Glycosylation Sites from SLX-2047 and SLX-2065

The light chain variable region (VL) of SLX-2047 and the heavy chain variable region (V_{H}) of SLX-2065 contained potential N-linked glycosylation sites (NXS/T). These residues were mutated to generate versions of SLX-2047 and SLX-2065 that did not contain the glycosylation sites. Specifically, residue asparagine 28 (Kabat numbering system) of SLX-2047 VL was mutated to serine (SEQ ID NO:9) and asparagine 82 of SLX-2065 V_{H} was mutated to lysine (SEQ ID NO: 13). The proteins were expressed and characterized.

### Example 4: Affinity Maturation of SLX-2079

Higher affinity variants of SLX-2079 were identified by synthesizing CDR libraries (all six CDRs were mutated individually, creating six libraries), and expressing and screening Fab fragments expressed using a phage expression system. The libraries were initially screened using an ELISA format. One hundred ninety clones displaying >2-fold increase in ELISA binding were sequenced. Ten mutations (five positions) were selected for synthesis of a combinatorial library that had a diversity of 216. Screening of the combinatorial library identified multiple clones with higher affinity to soluble antigen and tighter binding to live MIDA-MB-453 cells. The binding data for eight of these clones, as well as the parental antibody, SLX-2079, is shown in Table 5 (mutation residue number based on the Kabat numbering system).

TABLE 5
Characterization of Affinity Matured SLX-2079 Variants

| | | | | ELISA Antigen | | MDA-MB-453 | |
|---|---|---|---|---|---|---|---|
| | | | | Binding | | Binding | |
| | Mutations | | | Max | EC₅₀ | Max | EC₅₀ |
| Antibody | LCDR1 | HCDR2 | HCDR3 | OD450 | (nM) | MFI | (nM) |
| SLX-2079 | None | None | None | 3.123 | 2.844 | 2100 | 11.47 |
| SLX-2171 | None | None | F100aY | 3.088 | 0.3193 | 2100 | 8.044 |
| SLX-2172 | Q27Y | G54R | A98V, F100aY | 3.043 | 0.1081 | 2100 | 0.9216 |
| SLX-2173 | Q27Y | G54A | A98I, F100aW | 3.085 | 0.1111 | 2058 | 1.273 |
| SLX-2174 | Q27W | G54A | A98V | 3.046 | 0.1702 | 059 | 1.72 |
| SLX-2175 | Q27Y | G54M | A98I | 3.038 | 0.1864 | 2100 | 1.812 |
| SLX-2176 | None | G54A | A98I, F100aY | 3.083 | 0.1392 | 2040 | 2.222 |
| SLX-2177 | Q27W | None | A98V, F100aY | 3.009 | 0.209 | 2100 | 3.014 |
| SLX-2178 | None | G54M | F100aY | 3.048 | 0.2425 | 2099 | 5.215 |

### Example 5: Antibody Discovery Campaign 2

### Antibody Generation and Isolation

Two different strains of mice (NZB/W and CD) were immunized with various 5T4 antigens, as outlined in Table 6. 5T4 extra-cellular domain (amino acids 31-355) was from Bio-Techne, Cat. No. AV10290). Mouse B16 cells expressing two human/mouse chimeric 5T4 constructs were also used for immunization.

TABLE 6
Immunization Cohorts

| | Mouse | | SEQ |
|---|---|---|---|
| Cohort | Strain | Immunogen | ID NO: |
| A | NZB/W | 5T4 ECD residues 31-355 | 105 |
| B | NZB/W | B16 cells expressing human/mouse chimeric | 106 |
| | | 5T4 construct 1 | |
| C | NZB/W | B16 cells expressing human/mouse chimeric | 107 |
| | | 5T4 construct 2 | |
| D | CD1 | 5T4 ECD residues 31-355 | 105 |
| E | CD1 | B16 cells expressing human/mouse chimeric | 106 |
| | | 5T4 construct 1 | |
| F | CD1 | B16 cells expressing human/mouse chimeric | 107 |
| | | 5T4 construct 2 | |
| G | CD1 | 50% less 5T4 ECD residues 31-355 than | 105 |
| | | cohort D | |

Following immunization, antibodies were isolated using a traditional antibody hybridoma approach. Briefly, splenocytes were recovered from immunized mice and fused with a mouse myeloma cell line to produce hybridomas. Individual hybridoma clones were then isolated from the hybridoma libraries by limiting dilution and expanded in culture. Supernatants from these monoclonal hybridoma cultures were screened for antibodies with the ability to bind human 5T4 expressed on the surface of live cells expressing chimeric 5T4 constructs or human 5T4. Subsequently, hybridomas expressing anti-5T4 antibodies were sequenced to identify their variable heavy and light chain domains.

### Antibody Characterization

Hybridoma supernatants were screened for binding to live cells expressing various 5T4 constructs. The hybridoma supernatants were diluted 2-fold for the primary screen. Specifically, human/mouse chimeric 5T4 ECDs were expressed on mouse B16 cells. Construct 1 (SEQ ID NO: 106) consisted of amino-terminal human 5T4 sequence and membrane proximal mouse 5T4 sequence while construct 2 (SEQ ID NO: 107) consisted of amino-terminal mouse 5T4 sequence and membrane proximal human 5T4 sequence. Additionally, human 5T4 was expressed in HEK293T cells. Clones SLX-2104, SLX-2105 and SLX-2125 displayed stronger binding to chimeric construct 1, while clones SLX-2113 and SLX-2121 displayed stronger binding to chimeric construct 2 (Table 7). These data are consistent with clones SLX-2113 and SLX-2121 binding more proximal to the cell membrane than the other clones. As expected, all clones bound HEK293T cells expressing fully human 5T4.

The antibody concentration in the hybridoma supernatants was quantitated and the samples were characterized for binding to HEK293T-5T4 cells at 10 and 0.1 nM (Table 7).

Next, the antibodies were titrated on live MCF7 tumor cells and the binding EC₅₀ was determined. The antibodies bound live tumor cells tightly, with EC₅₀s ranging from 0.17 nM (SLX-2104) to 2.92 nM (SLX-2113).

TABLE 7
Binding Characterization of Hybridoma Supernatants

| | Primary Screen (Cells Expressing | | | | | | % |
|---|---|---|---|---|---|---|---|
| | Chimeric Constructs) | | | Secondary Screen | | MCF7 | Internalized |
| | Construct | Construct | HEK293T- | (HEK293T-5T4) | | EC₅₀ | (NCI-H226, |
| Antibody | 1 | 2 | 5T4 | 10 nM | 0.1 nM | (nM) | 4 h) |
| SLX-2104 | 91113 | 16721 | 231218 | 16814 | 11390 | 0.17 | 35 |
| SLX-2105 | 59416 | 14756 | 145604 | 9004 | 7514 | 0.92 | 35 |
| SLX-2113 | 10963 | 163178 | 194737 | 19279 | 1895 | 2.92 | 33 |
| SLX-2121 | 6055 | 145261 | 187004 | 32569 | 3009 | 0.82 | 35 |
| SLX-2125 | 111319 | 11986 | 85178 | 9481 | 3625 | 1.06 | 85 |

The internalization of the antibodies on NCI-H226 cells was characterized. Briefly, 1.5 μg/mL antibody was incubated with 5×10⁵ cells per sample on ice for 20 min with occasional gentle mixing. Unbound antibody was removed with three washes using ice-cold assay buffer (2% FBS in RPMI). Following the last wash, the cells were resuspended in ice-cold assay buffer and transferred to a 37° C. water bath for 4h. To terminate internalization, the cells were pelleted at 500 g for 3 min at 4° C., the supernatant was aspirated, and the cells were resuspended in 100 μL of secondary antibody (goat anti-human Fc PE, Invitrogen cat. #12-4998-82) and incubated on ice for 20 min. Samples were washed three times with 250 μL of ice-cold 2% FBS, 2 mM EDTA in PBS. For analysis of live cells, the cells were resuspended in 150 μL of 2% FBS, 2 mM EDTA in PBS and analyzed by flow cytometry. Alternatively, after the final wash the cells were fixed in 100 μL of paraformaldehyde in PBS for 10 min at 25° C. The cells were then washed once with 250 μL of 2% FBS, 2 mM EDTA in PBS, resuspended in 150 μL of 2% FBS, 2 mM EDTA in PBS and analyzed by flow cytometry. The binding signal obtained from cells incubated on ice the entire time (time 0) was designated 100% surface binding and internalization was quantitated by measuring the loss of binding signal following incubation at 37° C. for 4h.

Four of the antibodies (SLX-2104, SLX-2105, SLX-2113 and SLX-2121) displayed similar internalization properties, while one antibody (SLX-2125) internalized to a much greater extent (Table 7).

The binding kinetics of the antibodies were measured directly from hybridoma supernatants using a Gator biolayer interferometry (BLI) biosensor. Antibodies were captured from undiluted supernatants using anti-mouse Ig Fc capture sensors. After 700 seconds the loaded sensors were transferred to quenching buffer (containing 1000 nM whole mouse IgG) for 300 seconds to block free Fc capture sites. Next, sensors were transferred to equilibration buffer for 300 seconds. Following equilibration, sensors were transferred to a 20 nM solution of recombinant human 5T4 ECD (Bio-Techne, Cat. No. AVI10290) for 600 seconds to allow antibody/antigen association. The sensors were then transferred to equilibration buffer for 600 seconds to allow antibody/antigen dissociation. The association and dissociation phases were measured as nanometer shift in the BLI signal wavelength, and this data was fitted to a 1:1 kinetic binding model. The clones displayed affinities ranging from 0.8 nM (SLX-2105) to 11.2 nM (SLX-2121), as summarized in Table 8.

TABLE 8
Kinetic Binding Characterization of Hybridoma Supernatants

BLI Kinetics

| Antibody | Response (nm) | K_{D} (nM) |
|---|---|---|
| SLX-2104 | 0.096 | 4.3 |
| SLX-2105 | 0.215 | 0.8 |
| SLX-2113 | 0.188 | 4.3 |
| SLX-2121 | 0.188 | 11.2 |
| SLX-2125 | ND | ND |

ND: not determined

### Example 6: Humanization of SLX-2104

### Sequence Analysis, Template Selection, and Framework Mutations

The heavy and light chain variable region sequences (SEQ ID NOs:3 and 4) of murine antibody SLX-2104 were analyzed for potential post-translational modification sites and the CDRs were identified. Multiple heavy and light chain CDR definitions were determined, as shown in FIG. 2. For CDR grafting, the CDRs were defined as some combination of Kabat, Chothia and IMGT definitions. For humanization, the final row of tables in FIG. 2 (Grafted Seq) indicates the residues that were grafted into human germline frameworks.

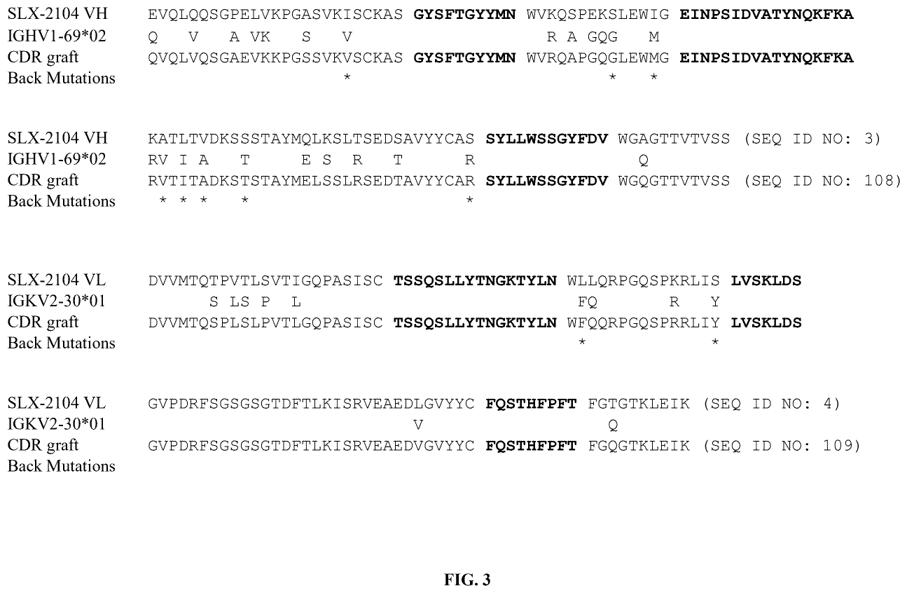

The most homologous human germline framework sequences were identified to be used as acceptor sequences for humanization. The sequence alignment of the heavy and light chains of SLX-2104 with the most homologous human germline heavy chain, IGHV1-69*02, and light chain, IGKV2-30*01, is shown in FIG. 3. Certain framework residues were identified as being potentially important for preserving the binding activity of SLX-2104 and these residues are marked with an asterisk in FIG. 3.

Multiple variants containing various combinations of back-mutations (human germline framework residues mutated back to corresponding murine framework residue) were synthesized and expressed as IgG_{I} (Z1 through Z16; FIG. 4). The positions of the mutations are indicated using the Kabat numbering system. A chimeric version of SLX-2104 was also made, with murine V_{H} and V_{L}, and human IgG_{I} and κ constant regions (ch2104). The framework (FR) back mutations and heavy and light chain combinations of the variants tested are summarized in FIG. 4. The variants were expressed transiently in Expi293F™ cells and were purified using Protein A chromatography.

In addition, a potential sequence liability was identified in SLX-2104. Specifically, there was a potential deamidation site identified in light chain CDR1 (Kabat numbering system, N28G29). Three variants that alter this site were expressed and characterized: p1 (N28Q), p2 (N28S), and p3 (G29A).

### Example 7: Characterization of Humanized Variants and Potential Deamidation Site Variants

All variants (p1, p2, p3, and humanization clones) were characterized for binding to recombinant human 5T4 and binding to a human tumor cell line expressing 5T4 (MDA-MB-453).

Binding of the variants to live MDA-MB-453 cells was evaluated using flow cytometry. Briefly, 100,000 cells/well were incubated with the antibody variants (diluted serially 5-fold in 1% BSA beginning at 100 nM) for 1h at 4° C. in a 96-well plate. Unbound antibody was removed by washes and the cells were incubated with R-phycoerythrin (RPE)-labeled anti-human Fc antibody (diluted 100-fold in 1% BSA) for 0.5h at 4° C. Unbound antibody was removed by washes and the cells were analyzed by flow cytometry. The top MFI and EC₅₀ for cell binding are shown in Table 9.

Binding of the purified variants to the extracellular domain (ECD) of recombinant human 5T4 (R&D Systems, cat. no. AVI10290) was characterized by SPR using a Biacore™ 8K. Antibody was captured using anti-human Fc IgG (Jackson ImmunoResearch) immobilized on a CM5 chip (Cytiva). The antibodies were diluted 6- to 30-fold and the contact time was 60 s at a flow rate of 10 μL/min. Recombinant 5T4 was used at a working concentration of 60 nM, with 120 s association time and a 300 s dissociation time at a flow rate of 30 μL/min. The running buffer was 10 mM HEPES, 150 mM NaCl, 3 mM EDTA, 0.05% Tween 20, pH 7.4. The binding data was evaluated using a 1:1 binding model and Biacore™ evaluation software. The kinetics and affinity of the variants are summarized in Table 9.

From these data, it was apparent that CDR-grafted antibody (Z0) displayed little binding to cells or recombinant antigen (Table 9).

TABLE 9
Binding of ch18327 and Humanized Variants to
Live MDA-MB-453 Tumor Cells and Human 5T4 ECD

| Ligand | Top MFI | EC₅₀ | kₐ (1/Ms) | k_{d} (1/s) | K_{D} (M) |
|---|---|---|---|---|---|
| Ch2104 | 1072 | 0.050 | 2.82E+05 | 9.84E−03 | 3.49E−08 |
| Z0 | NB | NB | NB | — | — |
| Z1 | 1071 | 0.022 | 2.91E+05 | 6.54E−03 | 2.25E−08 |
| Z2 | NB | NB | NB | — | — |
| Z3 | 834 | 0.062 | 2.22E+05 | 1.76E−02 | 7.93E−08 |
| Z4 | 1012 | 0.040 | 2.75E+05 | 9.37E−03 | 3.41E−08 |
| Z5 | 1032 | 0.048 | 2.48E+05 | 8.01E−03 | 3.23E−08 |
| Z6 | 960 | 0.047 | 2.10E+05 | 9.95E−03 | 4.74E−08 |
| Z7 | 1035 | 0.021 | 2.69E+05 | 9.37E−03 | 3.49E−08 |
| Z8 | 1050 | 0.021 | 2.79E+05 | 1.00E−02 | 3.60E−08 |
| Z9 | 1031 | 0.026 | 2.34E+05 | 9.24E−03 | 3.94E−08 |
| Z10 | 1018 | 0.044 | 2.33E+05 | 9.44E−03 | 4.06E−08 |
| Z11 | 985 | 0.060 | 1.88E+05 | 9.97E−03 | 5.30E−08 |
| Z12 | 851 | 0.108 | 1.67E+05 | 1.33E−02 | 7.93E−08 |
| Z13 | 778 | 0.122 | 1.21E+05 | 1.26E−02 | 1.04E−07 |
| Z14 | 807 | 0.152 | 1.04E+05 | 1.44E−02 | 1.38E−07 |
| Z15 | 648 | 0.317 | 1.30E+05 | 1.59E−02 | 1.22E−07 |
| Z16 | 1121 | 0.041 | 2.81E+05 | 9.06E−03 | 3.23E−08 |
| P1 | 1119 | 0.051 | 3.19E+05 | 1.02E−02 | 3.21E−08 |
| P2 | 1113 | 0.073 | 2.84E+05 | 9.34E−03 | 3.29E−08 |
| P3 | 1115 | 0.070 | 2.71E+05 | 9.48E−03 | 3.50E−08 |

NB: no or weak binding

Next several of the best humanized variants (based on maintenance of cell binding, affinity, and good expression; Z1, Z4, Z5, Z9) were combined with p1 and p2 variants to express humanized variants with the potential sequence liability removed. Humanized variant Z1 generally expressed 10-20-fold less protein than the other humanized variants. The binding of these variants to live MDA-MB-453 tumor cells and human 5T4 ECD is summarized in Table 10.

TABLE 10
Binding of Humanized/Optimized Variants to Live
MDA-MB-453 Tumor Cells and Human 5T4 ECD

| Ligand | Top MFI | EC50 | ka (1/Ms) | k_{d} (1/s) | K_{D} (M) |
|---|---|---|---|---|---|
| Ch2104 | 684 | 0.092 | 3.49E+05 | 8.92E−03 | 2.56E−08 |
| Z1 + p2 | 697 | 0.068 | 3.96E+05 | 6.75E−03 | 1.71E−08 |
| Z4 + p1 | 655 | 0.078 | 3.43E+05 | 9.85E−03 | 2.87E−08 |
| Z4 + p2 | 766 | 0.106 | 3.19E+05 | 8.81E−03 | 2.76E−08 |
| Z5 + p2 | 679 | 0.078 | 3.06E+05 | 7.56E−03 | 2.47E−08 |
| Z9 + p2 | 657 | 0.097 | 2.76E+05 | 8.89E−03 | 3.22E−08 |

Next, the thermal stability of select humanized/optimized variants was characterized by Differential Scanning Fluorimetry (DSF). Chimeric 2104 and five humanized/optimized variants were suspended in PBS at 1 mg/mL and the Tₘ₁ (° C.) was measured twice. The variants' mean Tₘ₁ ranged from 68.1° C. (Z1+p2) to 69.8° C. (Z9+p2), all somewhat lower than chimeric 2104 Tₘ₁ (73.5° C.), as summarized in Table 11.

A baculovirus particle (BVP) ELISA was employed to assess non-specific binding activity of select variants. Briefly, ELISA plates were coated with 1% BVP and antibody variants were incubated with the plate at 1 μM concentration. Unbound antibody was removed by washing and antibody binding was detected by incubating the plates with goat anti-human IgG Fc-HRP conjugate. Following removal of unbound antibody by washing, the plates were developed with HRP substrate and the A450 was determined. The BVP binding scores are summarized in Table 11. All humanized/optimized variants tested displayed less non-specific binding than the chimeric 2104 antibody. For reference, a positive control antibody had a BVP score of 62.95 and a negative control antibody had a BVP score of 1.00 in this assay.

TABLE 11
Thermal Stability of Humanized/Optimized Variants
to Live MDA-MB-453 Tumor Cells and Human 5T4 ECD

| Ligand | Average Tₘ₁ (° C.) | BVP Score |
|---|---|---|
| Ch2104 | 73.5 | 22.39 |
| Z1 + p2 | 68.1 | ND |
| Z4 + p1 | 68.7 | ND |
| Z4 + p2 | 69.3 | 8.40 |
| Z5 + p2 | 68.1 | 9.27 |
| Z9 + p2 | 69.8 | 9.26 |

ND: not determined

### Example 8: Internalization of Select 5T4 Antibodies

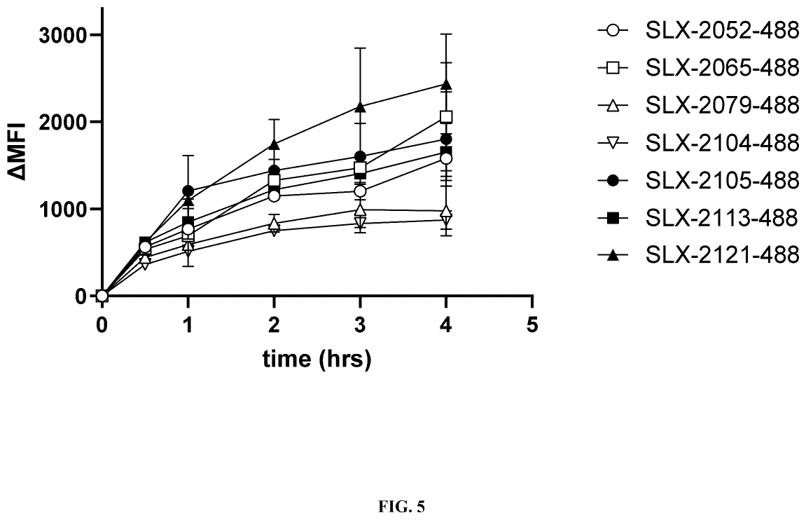

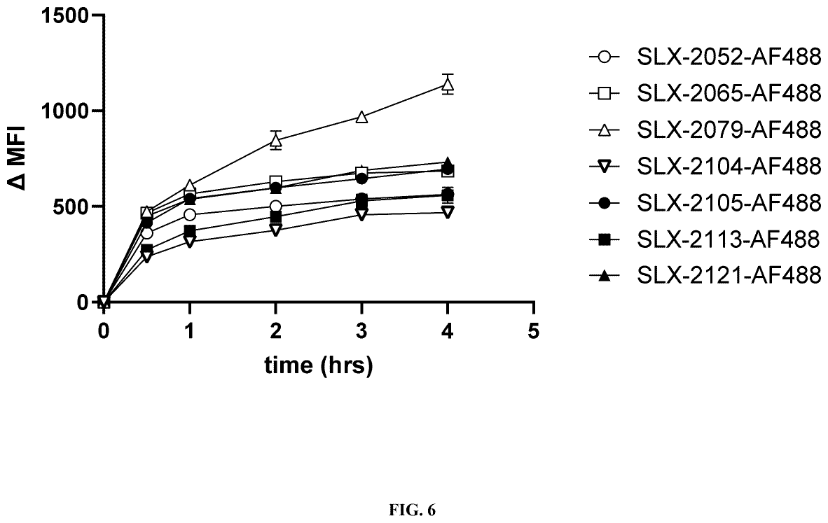

Select antibodies were characterized for internalization on two tumor cell lines, NCI-H226 (FIG. 5) and NCI-H520 (FIG. 6). Tumor cells in single-cell suspension were incubated with AlexaFluor 488 conjugated antibodies at a final concentration of 10 μg/mL on ice for 30 minutes. Labeled cells were transferred into a 200 μL tube and incubated at 37° C. for the indicated time lengths. After washes, cells were incubated with the rabbit anti-AlexaFluor 488 polyclonal Ab (ThermoFisher cat. no. 710369) at a final concentration of 15 μg/mL on ice for 30 minutes, resulting in quenching of Alexa Fluor 488 fluorescence on the cell surface. After washes, cells were fixed with 2% PFA and analyzed using a BD Fortessa. The median fluorescence intensity of AlexaFluor 488 in the quenched cells was measured as an indication of receptor internalization.

Interestingly, the antibodies behaved somewhat differently, depending on the cell line tested. For example, SLX-2079 internalized to a lesser extent than the other candidates (with the exception of SLX-2104) on NCI-H226 cells (FIG. 5), yet internalized to the greatest extent on NCI-H520 cells (FIG. 6). SLX-2104 internalized less than the other candidates on both cell lines. SLX-2121 was among the best internalizing antibodies on both cell lines.

### Sequences

The table below shows sequences described herein (SEQ: SEQ ID NO).

| Description | | SEQ | Sequence |
|---|---|---|---|
| SLX-2079 | V_{H} | 1 | QVQLQESGPGLVKPPGTLSLTCAVSGGSISSSNWWSWVRQPP |
| (Ab6, 5T4-7) | | | GKGLEWIGEIYHGGYTNYNPSLKSRVTISIDKSKNQFSLKLS |
| | | | SVTAADTAVYYCTRDMGANYFGSGNYYDVGWFDPWGQGTLVT |
| | | | VSS |
| | V_{L} | 2 | DIQLTQSPSFLSASVGDRVTITCRASQGIRSYLAWYQQKPGK |
| | | | APKVLIYEASTLQSGVPSRFSGSGSGTEFTLTISSLQPEDFA |
| | | | TYYCQQLNNYPRTFGQGTKVEIK |
| SLX-2104 | V_{H} | 3 | EVQLQQSGPELVKPGASVKISCKASGYSFTGYYMNWVKQSPE |
| (5T4-11) | | | KSLEWIGEINPSIDVATYNQKFKAKATLTVDKSSSTAYMQLK |
| | | | SLTSEDSAVYYCASSYLLWSSGYFDVWGAGTTVTVSS |
| | V_{L} | 4 | DVVMTQTPVTLSVTIGQPASISCTSSQSLLYTNGKTYLNWLL |
| | | | QRPGQSPKRLISLVSKLDSGVPDRFSGSGSGTDFTLKISRVE |
| | | | AEDLGVYYCFQSTHFPFTFGTGTKLEIK |
| SLX-2024 | V_{H} | 5 | QVQLQESGPGLVKPPGTLSLTCAVSGGSISSSNWWSWVRQPP |
| (5T4-2) | | | GKGLEWIGEIYQSGSTNYHPTLKSRVTISVDKSNNQFSLKLR |
| | | | SVTAADTAVYYCARDMGTYYYGSGNYYNVGWFDPWGQGTLVT |
| | | | VSS |
| | V_{L} | 6 | DIQLTQSPSFLSASVGDRVTITCRASQGISSYLAWYQQKPGK |
| | | | APKVLIYAASTLQSGVPSRFSGSGSGTEFTLTISSLQPEDFA |
| | | | TYYCQQLNYYPRTFGQGTKVEIK |
| SLX-2047 | V_{H} | 7 | EVQLVESGGGVVRPGGSLRLSCAASGFTEDDYGMSWVRQAPG |
| (5T4-3) | | | KGLEWVSGINWNGGSTVYADSVKGRFTISRDNAKNSLFLQMN |
| | | | SLRAEDTALYYCARGHFWSGPHDAFDIWGQGTMVTVSS |
| | V_{L} | 8 | DIQMTQSPSSLSASVGDRVTITCRASQNISTSLNWYQQKRGK |
| | | | APKLLVYTTSSLRSGVPSRFSGSGSGTDFTLTISSLQPEDFA |
| | | | TYFCQQNYRTPFTFGGGTKVEIK |
| | V_{L} | 9 | DIQMTQSPSSLSASVGDRVTITCRASQSISTSLNWYQQKRGK |
| | (N28S) | | APKLLVYTTSSLRSGVPSRFSGSGSGTDFTLTISSLQPEDFA |
| | | | TYFCQQNYRTPFTFGGGTKVEIK |
| SLX-2052 | V_{H} | 10 | EVQLVESGGGLVQPGGSLRLSCAASGFTFSSYSMNWVRQAPG |
| (5T4-4) | | | KGLEWVSYINSVSNTIYYADSVKGRFTISRDNAKNSLYLQMN |
| | | | SLRDEDTAVYYCARGGVRVLRSYFDLWGRGTLVTVSS |
| | V_{L} | 11 | DIQMTQSPSSLSASVGDRVTITCRASQTISSYLNWFQQKPGK |
| | | | APNLLIYTTSILQSGVPSRFSGSGSGTDFTLTISSLQPEDFA |
| | | | TYYCQQSYSTPLTFGGGTKVEIK |
| SLX-2065 | V_{H} | 12 | QVQLQESGPGLVKPPGTLSLTCAVSGGSISSSNWWTWVRQPP |
| (5T4-5) | | | GKGLEWIGEIFHSGFTNYNPSLKSRVTMSVDKSKKQFSLNLS |
| | | | SVTAADTAVYYCAREGLELGAFDIWGQGTMVTVSS |
| | V_{H} | 13 | QVQLQESGPGLVKPPGTLSLTCAVSGGSISSSNWWTWVRQPP |
| | (N82K) | | GKGLEWIGEIFHSGFTNYNPSLKSRVTMSVDKSKKQFSLKLS |
| | | | SVTAADTAVYYCAREGLELGAFDIWGQGTMVTVSS |
| | V_{L} | 14 | AIQMTQSPSSLSASVGDRVTITCRASQGIRNDLGWYQQKPGK |
| | | | APKVLIYGASSLQSGVPSRFSGSGSGTDFTLTISSLQPEDSG |
| | | | TYYCLQDNNYPRTFGQGTKVEIK |
| SLX-2077 | V_{H} | 15 | EVQLVESGGGLVQPGGSLRLSCAASGFTFSSRSMNWVRQAPG |
| (5T4-6) | | | RGLEWVSYIGFSSSTIYYVDSVKGRFTISRDNAKNSLYLQMN |
| | | | TLRDEDTAVYYCARGGVRYFDHPLDVWGKGTTVTVSS |
| | V_{L} | 16 | DIQMTQSPSTLSASVGDRVTITCRASQSISSWLAWYQQKPGK |
| | | | APKLLIYKASSLESGVPSRFSGSGSGTQFTLTISSLQPDDFA |
| | | | TYYCQQYYSYYTFGQGTKVEIK |
| SLX-2089 | V_{H} | 17 | QVQLVQSGAEVKKPGASVKVSCKASGYTFTTYDIIWVRQATG |
| (5T4-10) | | | QGLEWMGWMNPKSGNTGYAQKFQGRVSMTRNTSINTAYMELS |
| | | | TLRSEDTALYYCARGGLEPDYWGQGTLVTVSS |
| | V_{L} | 18 | DIQMTQSPSSLSASVGDRVTITCRASQGIRNDLAWFQQKPGK |
| | | | TPKRLIYDASNLQSGVPSRFSGSGSGTEFTLTIRSLQPEDFA |
| | | | TYYCLQYNRYPRTFGQGTKVEIK |
| SLX-2095 | V_{H} | 19 | QVQLVESGGGLVKPGGSLRLSCAASGFSISDYYMTWIRQAPG |
| (5T4-8) | | | KGLEWVTYISSSDTTIYYVDSVKGRFTISKDNAKNSLYLQMN |
| | | | SLRAEDTAVYYCAREKGYYYGSGSYYNRGWFDPWGQGTLVTV |
| | | | SS |
| | V_{L} | 20 | EIVMTQSPATLSVSPGERATLSCRASQSVTSNLAWYQQKPGQ |
| | | | APRLLIYGASTRATGIPARFSGSGSGTEFTLVISSLQSEDFA |
| | | | VYFCHQFNNWPLTFGGGTKVEIK |
| SLX-2105 | V_{H} | 21 | EVMLVESGGGLVKPGGSLKLSCAASGFTFSDYAMSWVRQTPE |
| (5T4-12) | | | KRLEWVAYISSGGGNTYYLDSVKGRFTISRDNAKNTLYLQMS |
| | | | SLRSEDTALFYCAREESYNYYRHFFDYWGQGTTLTVSS |
| | V_{L} | 22 | EIVLTQSPALMAASPGEKVTITCSVSSSISSSSLHWYQQKSG |
| | | | ASPKPWIYGTSNLASGVPVRFSGSGSGTSFSLTISSMEAEDA |
| | | | ATYYCQQWSSYPPILTFGAGTKLELK |
| SLX-2113 | V_{H} | 23 | QGQLQQSGAELMKPGASVKISCRGTGYTFRSYWIEWVKQRPG |
| (5T4-13) | | | HGLEWIGEILPGIGSTYYNEKFKGKATFTADTSSNTAYMQLS |
| | | | SLTSDDSAVYFCARGGRDFDYWGQGTTLTVSS |
| | V_{L} | 24 | DIQMTQTTSSLSASLGDRVTISCRASQDINNYLNWYQQKPDG |
| | | | TVKLLIYDTSRLHSGVPSRFSGSGSGTDFSLTISNLEPEDIA |
| | | | TYYCQQYSKLPYTFGSGTKLEIK |
| SLX-2121 | V_{H} | 25 | EVQLQQSGPELVKPGASVKMSCKASGFTFTNYVFHWVKQKPG |
| (5T4-14) | | | QGLEWIGYINPYNDGTKYNERFKGKATLTSDKSSSTAYMELS |
| | | | SLTSEDSAVYFCARVGNYVFAYWGQGTLVTVSA |
| | V_{L} | 26 | DIVLTQSPASLAVSLGQSVTISCRASESVEYYGTSLMQWYQQ |
| | | | KPGQPPKLLIYGASNVESGVPARFSGSGSGTDFSLNIHPVEE |
| | | | DDIAMYFCQQSRKVPWTFGGGTKLEIK |
| SLX-2125 | V_{H} | 27 | EVQLQQSGPELVKPGASVKISCKASGYSFTGYFLNWVKLSHG |
| (5T4-15) | | | KSLEWIGRINPYNGDTFYNQKFKGKATLTVDKSSSTAHMEVR |
| | | | SLTSEDSAVYYCARREGNLYSMDYWGQGTSVTVSS |
| | V_{L} | 28 | DIVMTQSPSSLTVTAGEKVTLSCKSSQSLLNSGNQKNELTWY |
| | | | QQKPGQPPKLLIYWASTRESGVPDRFTGSGSGTDFTLAISSV |
| | | | QAEDLAVYYCQNDYSYPLTFGAGTKLELK |
| SLX-2168 | V_{H} | 29 | QVQLVQSGAEVKKPGSSVKVSCKASGYSFTGYYMNWVRQAPG |
| (z4) | | | QGLEWMGEINPSIDVATYNQKFKARVTITVDKSTSTAYMELS |
| (5T4-16) | | | SLRSEDTAVYYCASSYLLWSSGYFDVWGQGTTVTVSS |
| | V_{L} | 30 | DVVMTQSPLSLPVTLGQPASISCTSSQSLLYTNGKTYLNWLQ |
| | | | QRPGQSPRRLISLVSKLDSGVPDRFSGSGSGTDFTLKISRVE |
| | | | AEDVGVYYCFQSTHFPFTFGQGTKLEIK |
| SLX-2169 | V_{H} | 31 | QVQLVQSGAEVKKPGSSVKVSCKASGYSFTGYYMNWVRQAPG |
| (z5) | | | QGLEWMGEINPSIDVATYNQKFKARATITVDKSTSTAYMELS |
| (5T4-17) | | | SLRSEDTAVYYCASSYLLWSSGYFDVWGQGTTVTVSS |
| | V_{L} | 32 | DVVMTQSPLSLPVTLGQPASISCTSSQSLLYTNGKTYLNWLQ |
| | | | QRPGQSPRRLISLVSKLDSGVPDRFSGSGSGTDFTLKISRVE |
| | | | AEDVGVYYCFQSTHFPFTFGQGTKLEIK |
| SLX-2170 | V_{H} | 33 | QVQLVQSGAEVKKPGSSVKVSCKASGYSFTGYYMNWVRQAPG |
| (z9) | | | QSLEWMGEINPSIDVATYNQKFKARVTITVDKSTSTAYMELS |
| (5T4-18) | | | SLRSEDTAVYYCASSYLLWSSGYFDVWGQGTTVTVSS |
| | V_{L} | 34 | DVVMTQSPLSLPVTLGQPASISCTSSQSLLYTNGKTYLNWLQ |
| | | | QRPGQSPRRLISLVSKLDSGVPDRFSGSGSGTDFTLKISRVE |
| | | | AEDVGVYYCFQSTHFPFTFGQGTKLEIK |
| SLX-2171 | V_{H} | 35 | QVQLQESGPGLVKPPGTLSLTCAVSGGSISSSNWWSWVRQPP |
| (5T4-19) | | | GKGLEWIGEIYHGGYTNYNPSLKSRVTISIDKSKNQFSLKLS |
| | | | SVTAADTAVYYCTRDMGANYYGSGNYYDVGWFDPWGQGTLVT |
| | | | VSS |
| | V_{L} | 36 | DIQLTQSPSFLSASVGDRVTITCRASQGIRSYLAWYQQKPGK |
| | | | APKVLIYEASTLQSGVPSRFSGSGSGTEFTLTISSLQPEDFA |
| | | | TYYCQQLNNYPRTFGQGTKVEIK |
| SLX-2176 | V_{H} | 37 | QVQLQESGPGLVKPPGTLSLTCAVSGGSISSSNWWSWVRQPP |
| (5T4-20) | | | GKGLEWIGEIYHAGYTNYNPSLKSRVTISIDKSKNQFSLKLS |
| | | | SVTAADTAVYYCTRDMGINYYGSGNYYDVGWFDPWGQGTLVT |
| | | | VSS |
| | V_{L} | 38 | DIQLTQSPSFLSASVGDRVTITCRASQGIRSYLAWYQQKPGK |
| | | | APKVLIYEASTLQSGVPSRFSGSGSGTEFTLTISSLQPEDFA |
| | | | TYYCQQLNNYPRTFGQGTKVEIK |
| SLX-2178 | V_{H} | 39 | QVQLQESGPGLVKPPGTLSLTCAVSGGSISSSNWWSWVRQPP |
| (5T4-21) | | | GKGLEWIGEIYHMGYTNYNPSLKSRVTISIDKSKNQFSLKLS |
| | | | SVTAADTAVYYCTRDMGANYYGSGNYYDVGWFDPWGQGTLVT |
| | | | VSS |
| | V_{L} | 40 | DIQLTQSPSFLSASVGDRVTITCRASQGIRSYLAWYQQKPGK |
| | | | APKVLIYEASTLQSGVPSRFSGSGSGTEFTLTISSLQPEDFA |
| | | | TYYCQQLNNYPRTFGQGTKVEIK |
| SLX-2001 | V_{H} | 41 | EVQLVESGGGLVQPGGSLRLSCAASGYTFTNFGMNWVRQAPG |
| | | | KGLEWVAWINTNTGEPRYAEEFKGRFTISRDNAKNSLYLQMN |
| | | | SLRAEDTAVYYCARDWDGAYFFDYWGQGTLVTVSS |
| | V_{L} | 42 | DIQMTQSPSSLSASVGDRVTITCKASQSVSNDVAWYQQKPGK |
| | | | APKLLIYFATNRYTGVPSRESGSGYGTDFTLTISSLQPEDFA |
| | | | TYYCQQDYSSPWTFGQGTKVEIK |
| SLX-2002 | VH | 43 | EVQLEESGGGLVKPGGSLRLSCAASGIDLSHYVVGWVRQAPG |
| | | | KGLEWVSIIYGSGRTYYANWAKGRFTISRDNSKNTLYLQMNS |
| | | | LRAEDTAVYYCARDASVSVYYWGYFDLWGRGTLVTVSS |
| | V_{L} | 44 | DIQMTQSPSTLSASVGDRVTITCQASQSIGSELAWYQQKPGK |
| | | | APKLLIYRASTLESGVPSRFSGSGSGTEFTLTISSLQPDDFA |
| | | | TYYCQQGYTYSEIDNAFGQGTKVEIK |
| SLX-2003 | V_{H} | 45 | EVQLVESGGDLAQPGGSLRLSCAVSGIDLSSYGMGWVRQAPG |
| | | | KGLEWVSIISRNSVTYYATWAKGRFTISRDNSKNTVYLQMTS |
| | | | LRAEDTALYFCARRATYSGALGYFDIWGQGTLVTVSS |
| | V_{L} | 46 | EIVMTQSPSSLSASVGDRVTITCQASENIYSTLAWYQQKPGK |
| | | | APKLLIYDAFDLASGVPSRFSGSGSGTDYTLTISSLQPEDFA |
| | | | TYYCQQGYSGTNVDNAFGQGTKLEIK |

SLX-2079 CDR Sequences

| | | | |
|---|---|---|---|
| HCDR1 | IMGT | 47 | GGSISSSNW |
| | Kabat | 48 | SSNWWS |
| | Chothia | 49 | GGSISSSN |
| | AbM | 50 | GGSISSSNWWS |
| | Contact | 51 | SSSNWWS |
| HCDR2 | IMGT | 52 | IYHGGYT |
| | Kabat | 53 | EIYHGGYTNYNPSLKS |
| | Chothia | 54 | YHGGY |
| | AbM | 55 | EIYHGGYTN |
| | Contact | 56 | WIGEIYHGGYTN |
| HCDR3 | IMGT | 57 | TRDMGANYFGSGNYYDVGWFDP |
| | Kabat/ | 58 | DMGANYFGSGNYYDVGWFDP |
| | Chothia/ | | |
| | AbM | | |
| | Contact | 59 | TRDMGANYFGSGNYYDVGWFD |
| LCDR1 | IMGT | 60 | QGIRSY |
| | Kabat/ | 61 | RASQGIRSYLA |
| | Chothia/ | | |
| | AbM | | |
| | Contact | 62 | RSYLAWY |
| LCDR2 | IMGT | 63 | EA |
| | Kabat/ | 64 | EASTLQS |
| | Chothia/ | | |
| | AbM | | |
| | Contact | 65 | VLIYEASTLQ |
| LCDR3 | IMGT/ | 66 | QQLNNYPRT |
| | Kabat/ | | |
| | Chothia/ | | |
| | AbM | | |
| | Contact | 67 | QQLNNYPR |

SLX-2104 CDR Sequences

| | | | |
|---|---|---|---|
| HCDR1 | IMGT | 68 | GYSFTGYY |
| | Kabat | 69 | GYYMN |
| | Chothia | 70 | GYSFTGY |
| | AbM | 71 | GYSFTGYYMN |
| | Contact | 72 | TGYYMN |
| | Grafted | 73 | GYSFTGYYMN |
| HCDR2 | IMGT | 74 | INPSIDVA |
| | Kabat/ | 75 | EINPSIDVATYNQKFKA |
| | Grafted | | |
| | Chothia | 76 | NPSIDV |
| | AbM | 77 | EINPSIDVAT |
| | Contact | 78 | WIGEINPSIDVAT |
| HCDR3 | IMGT | 79 | ASSYLLWSSGYFDV |
| | Kabat/ | 80 | SYLLWSSGYFDV |
| | Chothia/ | | |
| | AbM/ | | |
| | Grafted | | |
| | Contact | 81 | ASSYLLWSSGYFD |
| LCDR1 | IMGT | 82 | QSLLYTNGKTY |
| | Kabat/ | 83 | TSSQSLLYTNGKTYLN |
| | Chothia/ | | |
| | AbM/ | | |
| | Grafted | | |
| | Contact | 84 | LYTNGKTYLNWL |
| LCDR2 | IMGT | 85 | LV |
| | Kabat/ | 86 | LVSKLDS |
| | Chothia/ | | |
| | AbM/ | | |
| | Grafted | | |
| | Contact | 87 | RLISLVSKLD |
| LCDR3 | IMGT/ | 88 | FQSTHEPFT |
| | Kabat/ | | |
| | Chothia/ | | |
| | AbM/ | | |
| | Grafted | | |
| | Contact | 89 | FQSTHEPF |

SLX-2079 High Affinity Variant CDR Sequences (IMGT ®)

| | | | |
|---|---|---|---|
| G54A | HCDR2 | 90 | IYHAGYT |
| substitution | | | |
| SLX-2173 | | | |
| SLX-2174 | | | |
| SLX-2176 | | | |
| G54M | HCDR2 | 91 | IYHMGYT |
| substitution | | | |
| SLX-2175 | | | |
| SLX-2178 | | | |
| G54R | HCDR2 | 92 | IYHRGYT |
| substitution | | | |
| SLX-2172 | | | |
| A98I | HCDR3 | 93 | TRDMGINYFGSGNYYDVGWFDP |
| substitution | | | |
| SLX-2175 | | | |
| A98V | HCDR3 | 94 | TRDMGVNYFGSGNYYDVGWFDP |
| substitution | | | |
| SLX-2174 | | | |
| F100aY | HCDR3 | 95 | TRDMGANYYGSGNYYDVGWFDP |
| substitution | | | |
| SLX-2171 | | | |
| A98I, | HCDR3 | 96 | TRDMGINYWGSGNYYDVGWFDP |
| F100aW | | | |
| substitutions | | | |
| SLX-2173 | | | |
| A98I, | HCDR3 | 97 | TRDMGINYYGSGNYYDVGWFDP |
| F100aY | | | |
| substitutions | | | |
| SLX-2176 | | | |
| A98V, | LCDR1 | 98 | TRDMGVNYYGSGNYYDVGWFDP |
| F100aY | | | |
| substitutions | | | |
| SLX-2172 | | | |
| SLX-2177 | | | |
| Q27W | LCDR1 | 99 | WGIRSY |
| substitution | | | |
| SLX-2174 | | | |
| SLX-2177 | | | |
| Q27Y | LCDR1 | 100 | YGIRSY |
| substitution | | | |
| SLX-2172 | | | |
| SLX-2173 | | | |
| SLX-2175 | | | |

Other Sequences

| Human Heavy Chain | 101 | ASTKGPSVFPLAPSSKSTSGGTAALGCLVKDYFPEPVTVSWN |
|---|---|---|
| Constant Region | | SGALTSGVHTFPAVLQSSGLYSLSSVVTVPSSSLGTQTYICN |
| | | VNHKPSNTKVDKKVEPKSCDKTHTCPPCPAPELLGGPSVFLF |
| | | PPKPKDTLMISRTPEVTCVVVDVSHEDPEVKFNWYVDGVEVH |
| | | NAKTKPREEQYNSTYRVVSVLTVLHQDWLNGKEYKCKVSNKA |
| | | LPAPIEKTISKAKGQPREPQVYTLPPSRDELTKNQVSLTCLV |
| | | KGFYPSDIAVEWESNGQPENNYKTTPPVLDSDGSFFLYSKLT |
| | | VDKSRWQQGNVFSCSVMHEALHNHYTQKSLSLSPGK |
| Human Effectorless | 102 | ASTKGPSVFPLAPSSKSTSGGTAALGCLVKDYFPEPVTVSWN |
| Heavy Chain Constant | | SGALTSGVHTFPAVLQSSGLYSLSSVVTVPSSSLGTQTYICN |
| Region | | VNHKPSNTKVDKKVEPKSCDKTHTCPPCPAPEAAGGPSVFLF |
| | | PPKPKDTLMISRTPEVTCVVVDVSHEDPEVKFNWYVDGVEVH |
| | | NAKTKPREEQYNSTYRVVSVLTVLHQDWLNGKEYKCKVSNKA |
| | | LAAPIEKTISKAKGQPREPQVYTLPPSRDELTKNQVSLTCLV |
| | | KGFYPSDIAVEWESNGQPENNYKTTPPVLDSDGSFFLYSKLT |
| | | VDKSRWQQGNVFSCSVMHEALHNHYTQKSLSLSPGK |
| Human Light Chain | 103 | RTVAAPSVFIFPPSDEQLKSGTASVVCLLNNFYPREAKVQWK |
| Constant Region | | VDNALQSGNSQESVTEQDSKDSTYSLSSTLTLSKADYEKHKV |
| | | YACEVTHQGLSSPVTKSFNRGEC |
| Human 5T4 | 104 | MPGGCSRGPAAGDGRLRLARLALVLLGWVSSSSPTSSASSES |
| | | SSAPFLASAVSAQPPLPDQCPALCECSEAARTVKCVNRNLTE |
| | | VPTDLPAYVRNLFLTGNQLAVLPAGAFARRPPLAELAALNLS |
| | | GSRLDEVRAGAFEHLPSLRQLDLSHNPLADLSPFAFSGSNAS |
| | | VSAPSPLVELILNHIVPPEDERQNRSFEGMVVAALLAGRALQ |
| | | GLRRLELASNHFLYLPRDVLAQLPSLRHLDLSNNSLVSLTYV |
| | | SFRNLTHLESLHLEDNALKVLHNGTLAELQGLPHIRVFLDNN |
| | | PWVCDCHMADMVTWLKETEVVQGKDRLTCAYPEKMRNRVLLE |
| | | LNSADLDCDPILPPSLQTSYVELGIVLALIGAIFLLVLYLNR |
| | | KGIKKWMHNIRDACRDHMEGYHYRYEINADPRLTNLSSNSDV |
| Human 5T4 ECD | 105 | SSSPTSSASSESSSAPFLASAVSAQPPLPDQCPALCECSEAA |
| | | RTVKCVNRNLTEVPTDLPAYVRNLELTGNQLAVLPAGAFARR |
| | | PPLAELAALNLSGSRLDEVRAGAFEHLPSLRQLDLSHNPLAD |
| | | LSPFAFSGSNASVSAPSPLVELILNHIVPPEDERQNRSFEGM |
| | | VVAALLAGRALQGLRRLELASNHFLYLPRDVLAQLPSLRHLD |
| | | LSNNSLVSLTYVSFRNLTHLESLHLEDNALKVLHNGTLAELQ |
| | | GLPHIRVELDNNPWVCDCHMADMVTWLKETEVVQGKDRLTCA |
| | | YPEKMRNRVLLELNSADLDCDPILPPSLQTS |
| Human/Mouse 5T4 | 106 | MPGGCSRGPAAGDGRLRLARLALVLLGWVSSSSPTSSASSES |
| Chimeric | | SSAPFLASAVSAQPPLPDQCPALCECSEAARTVKCVNRNLTE |
| Construct 1 | | VPTDLPAYVRNLFLTGNQLAVLPAGAFARRPPLAELAALNLS |
| | | GSRLDEVRAGAFEHLPSLRQLDLSHNPLADLSPFAFSGSNAS |
| | | VSAPSPLVELILNHIVPPEDERQNRSFEGMVVAALRSGLALR |
| | | GLTRLELASNHFLFLPRDLLAQLPSLRYLDLRNNSLVSLTYA |
| | | SFRNLTHLESLHLEDNALKVLHNSTLAEWHGLAHVKVELDNN |
| | | PWVCDCYMADMVAWLKETEVVPDKARLTCAFPEKMRNRGLLD |
| | | LNSSDLDCDAVLPQSLQTSYVELGIVLALIGAIFLLVLYLNR |
| | | KGIKKWMHNIRDACRDHMEGYHYRYEINADPRLTNLSSNSDV |
| Human/Mouse 5T4 | 107 | MPGAGSRGPSAGDGRLRLARLALVLLGWVSASAPSSSVPSSS |
| Chimeric | | TSPAAFLASGSAQPPPAERCPAACECSEAARTVKCVNRNLLE |
| Construct 2 | | VPADLPPYVRNLELTGNQMTVLPAGAFARQPPLADLEALNLS |
| | | GNHLKEVCAGAFEHLPGLRRLDLSHNPLTNLSAFAFAGSNAS |
| | | VSAPSPLEELILNHIVPPEDQRQNGSFEGMVAFEGMVAAALL |
| | | AGRALQGLRRLELASNHFLYLPRDVLAQLPSLRHLDLSNNSL |
| | | VSLTYVSFRNLTHLESLHLEDNALKVLHNGTLAELQGLPHIR |
| | | VFLDNNPWVCDCHMADMVTWLKETEVVQGKDRLTCAYPEKMR |
| | | NRVLLELNSADLDCDPILPPSLQTSYVELGIVLALIGAIFLL |
| | | VLYLNRKGIKKWMHNIRDACRDHMEGYHYRYEINADPRLTNL |
| | | SSNSDV |

| | | |
|---|---|---|
| humanized SLX-2104 | 108 | QVQLVQSGAEVKKPGSSVKVSCKASGYSFTGYYMNWVRQAPG |
| VH with CDR graft | | QGLEWMGEINPSIDVATYNQKFKARVTITADKSTSTAYMELS |
| (FIG. 3) | | SLRSEDTAVYYCARSYLLWSSGYFDVWGQGTTVTVSSDVVMT |
| | | QSPLSLPVTLGQPASISCTSSQSLLYTNGKTYLNWFQQRPGQ |
| | | SPRRLIYLVSKLDSGVPDRFSGSGSGTDFTLKISRVEAEDVG |
| | | VYYCFQSTHFPFTFGQGTKLEIK |
| humanized SLX-2104 | 109 | DVVMTQSPLSLPVTLGQPASISCTSSQSLLYTNGKTYLNWFQ |
| VL with CDR graft | | QRPGQSPRRLIYLVSKLDSGVPDRFSGSGSGTDFTLKISRVE |
| (FIG. 3) | | AEDVGVYYCFQSTHFPFTFGQGTKLEIK |

1. An isolated anti-5T4 antibody or an antigen-binding portion thereof, wherein said antibody binds to the same epitope as, or competes for binding to human 5T4 with, an antibody comprising a heavy chain variable domain (V_{H}) and a light chain variable domain (V_{L}) that comprise SEQ ID NOs:1 and 2, respectively.

2. The isolated antibody or antigen-binding portion of claim 1, wherein said antibody comprises
a) heavy chain CDR (HCDR) 1-3 and light chain CDR (LCDR) 1-3 comprising SEQ ID NOs:47, 52, 57, 60, 63, and 66, respectively; or
b) a V_{H} and a V_{L} comprising SEQ ID NOs:1 and 2, respectively.

3. (canceled)

4. An isolated anti-5T4 antibody or an antigen-binding portion thereof, wherein said antibody binds to the same epitope as, or competes for binding to human 5T4 with, an antibody comprising a heavy chain variable domain (V_{H}) and a light chain variable domain (V_{L}) that comprise SEQ ID NOs:3 and 4, respectively.

5. The isolated antibody or antigen-binding portion of claim 4, wherein said antibody comprises
a) heavy chain CDR (HCDR) 1-3 and light chain CDR (LCDR) 1-3 comprising SEQ ID NOs:68, 74, 79, 82, 85, and 88, respectively; or
b) a V_{H} and a V_{L} comprising SEQ ID NOs:3 and 4, respectively.

6. (canceled)

7. The isolated antibody or antigen-binding portion of claim 4, wherein said antibody comprises a V_{H} comprising SEQ ID NOs:73, 75, and 80, and a V_{L} comprising SEQ ID NOs:83, 86, and 88.

8. The isolated antibody or antigen-binding portion of claim 7, wherein the antibody or antigen-binding portion is humanized.

9. The isolated antibody or antigen-binding portion of claim 8, wherein
a) the V_{H} utilizes framework regions from a human IGHV1-69*02 germline gene and optionally comprises one or more back mutations selected from V20I, G44S, M48I, V67A, I69L, A71V, T75S, and R94S, wherein the numbering is according to Kabat; and/or
b) the V_{L} utilizes framework regions from a human IGKV2-30*01 germline gene and optionally comprises one or more back mutations selected from F36L and Y49S, wherein the numbering is according to Kabat.

10-12. (canceled)

13. The isolated antibody or antigen-binding portion of claim 4, wherein
a) the V_{H} comprises any one of SEQ ID NOs:3, 29, 31, and 33, or an amino acid sequence at least 95% identical thereto;
b) the V_{L} comprises any one of SEQ ID NOs:4, 30, 32, and 34, or an amino acid sequence at least 95% identical thereto; or
c) a) and b).

14. The isolated antibody or antigen-binding portion of claim 13, wherein the V_{H} and the V_{L} are at least 95% identical in sequence to, or optionally comprise:
SEQ ID NOs:3 and 4, respectively,
SEQ ID NOs:29 and 30, respectively,
SEQ ID NOs:31 and 32, respectively, or
SEQ ID NOs:33 and 34, respectively.

15. (canceled)

16. The isolated antibody or antigen-binding portion of claim 2, wherein the antibody comprises a human IgG₁ constant region, optionally wherein the human IgG₁ constant region comprises SEQ ID NO:102, with or without the C-terminal lysine.

17. (canceled)

18. The isolated antibody or antigen-binding portion of claim 2, wherein the antibody comprises a human light chain constant region that comprises SEQ ID NO:103.

19. An isolated anti-5T4 antibody comprising
a) a heavy chain that comprises the amino acid sequences of SEQ ID NOs:1 and 102, with or without the C-terminal lysine of SEQ ID NO:102, and a light chain that comprises the amino acid sequences of SEQ ID NOs:2 and 103,
b) a heavy chain that comprises the amino acid sequences of SEQ ID NOs:3 and 102, with or without the C-terminal lysine, and a light chain that comprises the amino acid sequences of SEQ ID NOs:4 and 103;
c) a heavy chain that comprises the amino acid sequences of SEQ ID NOs:29 and 102, with or without the C-terminal lysine of SEQ ID NO:102, and a light chain that comprises the amino acid sequences of SEQ ID NOs:30 and 103;
d) a heavy chain that comprises the amino acid sequences of SEQ ID NOs:31 and 102, with or without the C-terminal lysine of SEQ ID NO:102, and a light chain that comprises the amino acid sequences of SEQ ID NOs:32 and 103; or
e) a heavy chain that comprises the amino acid sequences of SEQ ID NOs:33 and 102, with or without the C-terminal lysine of SEQ ID NO:102, and a light chain that comprises the amino acid sequences of SEQ ID NOs:34 and 103.

20-23. (canceled)

24. The isolated antibody or antigen-binding portion of claim 2, wherein the antibody or antigen-binding portion is conjugated to a cytotoxin or a detectable label.

25. A pharmaceutical composition comprising the isolated antibody or antigen-binding portion of claim 2 and a pharmaceutically acceptable excipient.

26. Nucleic acid molecule(s) encoding the isolated antibody or antigen-binding portion of claim 2.

27. Expression vector(s) comprising the nucleic acid molecule(s) of claim 26.

28. A recombinant host cell comprising the expression vector(s) of claim 27.

29. A method of making an anti-5T4 antibody or an antigen-binding portion thereof, comprising:
culturing the recombinant host cell of claim 28 under conditions that allow expression of the antibody or antigen-binding portion, and
isolating the antibody or antigen-binding portion from the cell culture.

30. A method of treating a 5T4-positive cancer in a patient in need thereof, comprising administering to the patient the isolated antibody or antigen-binding portion of claim 2.

31-32. (canceled)

33. The method of claim 30, wherein the 5T4-positive cancer is selected from adenocarcinoma, bladder cancer, bowel cancer, breast cancer, cervical cancer, colorectal cancer, esophageal cancer, gastric cancer, head and neck cancer, lung cancer, mesothelioma, ovarian cancer, pancreatic cancer, prostate cancer, renal cancer, squamous cell carcinoma, uterine cancer, retinoblastoma, and Ewing sarcoma.


SEQUENCE LISTING (109 sequences)

SEQ ID NO: 1 - protein, 129 aa, synthetic construct
QVQLQESGPGLVKPPGTLSLTCAVSGGSISSSNWWSWVRQPPGKGLEWIGEIYHGGYTNYNPSLKSRVTISIDKSKNQFSLKLSSVTAADTAVYYCTRDMGANYFGSGNYYDVGWFDPWGQGTLVTVSS

SEQ ID NO: 2 - protein, 107 aa, synthetic construct
DIQLTQSPSFLSASVGDRVTITCRASQGIRSYLAWYQQKPGKAPKVLIYEASTLQSGVPSRFSGSGSGTEFTLTISSLQPEDFATYYCQQLNNYPRTFGQGTKVEIK

SEQ ID NO: 3 - protein, 121 aa, synthetic construct
EVQLQQSGPELVKPGASVKISCKASGYSFTGYYMNWVKQSPEKSLEWIGEINPSIDVATYNQKFKAKATLTVDKSSSTAYMQLKSLTSEDSAVYYCASSYLLWSSGYFDVWGAGTTVTVSS

SEQ ID NO: 4 - protein, 112 aa, synthetic construct
DVVMTQTPVTLSVTIGQPASISCTSSQSLLYTNGKTYLNWLLQRPGQSPKRLISLVSKLDSGVPDRFSGSGSGTDFTLKISRVEAEDLGVYYCFQSTHFPFTFGTGTKLEIK

SEQ ID NO: 5 - protein, 129 aa, synthetic construct
QVQLQESGPGLVKPPGTLSLTCAVSGGSISSSNWWSWVRQPPGKGLEWIGEIYQSGSTNYHPTLKSRVTISVDKSNNQFSLKLRSVTAADTAVYYCARDMGTYYYGSGNYYNVGWFDPWGQGTLVTVSS

SEQ ID NO: 6 - protein, 107 aa, synthetic construct
DIQLTQSPSFLSASVGDRVTITCRASQGISSYLAWYQQKPGKAPKVLIYAASTLQSGVPSRFSGSGSGTEFTLTISSLQPEDFATYYCQQLNYYPRTFGQGTKVEIK

SEQ ID NO: 7 - protein, 122 aa, synthetic construct
EVQLVESGGGVVRPGGSLRLSCAASGFTFDDYGMSWVRQAPGKGLEWVSGINWNGGSTVYADSVKGRFTISRDNAKNSLFLQMNSLRAEDTALYYCARGHFWSGPHDAFDIWGQGTMVTVSS

SEQ ID NO: 8 - protein, 107 aa, synthetic construct
DIQMTQSPSSLSASVGDRVTITCRASQNISTSLNWYQQKRGKAPKLLVYTTSSLRSGVPSRFSGSGSGTDFTLTISSLQPEDFATYFCQQNYRTPFTFGGGTKVEIK

SEQ ID NO: 9 - protein, 107 aa, synthetic construct
DIQMTQSPSSLSASVGDRVTITCRASQSISTSLNWYQQKRGKAPKLLVYTTSSLRSGVPSRFSGSGSGTDFTLTISSLQPEDFATYFCQQNYRTPFTFGGGTKVEIK

SEQ ID NO: 10 - protein, 121 aa, synthetic construct
EVQLVESGGGLVQPGGSLRLSCAASGFTFSSYSMNWVRQAPGKGLEWVSYINSVSNTIYYADSVKGRFTISRDNAKNSLYLQMNSLRDEDTAVYYCARGGVRVLRSYFDLWGRGTLVTVSS

SEQ ID NO: 11 - protein, 107 aa, synthetic construct
DIQMTQSPSSLSASVGDRVTITCRASQTISSYLNWFQQKPGKAPNLLIYTTSILQSGVPSRFSGSGSGTDFTLTISSLQPEDFATYYCQQSYSTPLTFGGGTKVEIK

SEQ ID NO: 12 - protein, 119 aa, synthetic construct
QVQLQESGPGLVKPPGTLSLTCAVSGGSISSSNWWTWVRQPPGKGLEWIGEIFHSGFTNYNPSLKSRVTMSVDKSKKQFSLNLSSVTAADTAVYYCAREGLELGAFDIWGQGTMVTVSS

SEQ ID NO: 13 - protein, 119 aa, synthetic construct
QVQLQESGPGLVKPPGTLSLTCAVSGGSISSSNWWTWVRQPPGKGLEWIGEIFHSGFTNYNPSLKSRVTMSVDKSKKQFSLKLSSVTAADTAVYYCAREGLELGAFDIWGQGTMVTVSS

SEQ ID NO: 14 - protein, 107 aa, synthetic construct
AIQMTQSPSSLSASVGDRVTITCRASQGIRNDLGWYQQKPGKAPKVLIYGASSLQSGVPSRFSGSGSGTDFTLTISSLQPEDSGTYYCLQDNNYPRTFGQGTKVEIK

SEQ ID NO: 15 - protein, 121 aa, synthetic construct
EVQLVESGGGLVQPGGSLRLSCAASGFTFSSRSMNWVRQAPGRGLEWVSYIGFSSSTIYYVDSVKGRFTISRDNAKNSLYLQMNTLRDEDTAVYYCARGGVRYFDHPLDVWGKGTTVTVSS

SEQ ID NO: 16 - protein, 106 aa, synthetic construct
DIQMTQSPSTLSASVGDRVTITCRASQSISSWLAWYQQKPGKAPKLLIYKASSLESGVPSRFSGSGSGTQFTLTISSLQPDDFATYYCQQYYSYYTFGQGTKVEIK

SEQ ID NO: 17 - protein, 116 aa, synthetic construct
QVQLVQSGAEVKKPGASVKVSCKASGYTFTTYDIIWVRQATGQGLEWMGWMNPKSGNTGYAQKFQGRVSMTRNTSINTAYMELSTLRSEDTALYYCARGGLEPDYWGQGTLVTVSS

SEQ ID NO: 18 - protein, 107 aa, synthetic construct
DIQMTQSPSSLSASVGDRVTITCRASQGIRNDLAWFQQKPGKTPKRLIYDASNLQSGVPSRFSGSGSGTEFTLTIRSLQPEDFATYYCLQYNRYPRTFGQGTKVEIK

SEQ ID NO: 19 - protein, 128 aa, synthetic construct
QVQLVESGGGLVKPGGSLRLSCAASGFSISDYYMTWIRQAPGKGLEWVTYISSSDTTIYYVDSVKGRFTISKDNAKNSLYLQMNSLRAEDTAVYYCAREKGYYYGSGSYYNRGWFDPWGQGTLVTVSS

SEQ ID NO: 20 - protein, 107 aa, synthetic construct
EIVMTQSPATLSVSPGERATLSCRASQSVTSNLAWYQQKPGQAPRLLIYGASTRATGIPARFSGSGSGTEFTLVISSLQSEDFAVYFCHQFNNWPLTFGGGTKVEIK

SEQ ID NO: 21 - protein, 122 aa, synthetic construct
EVMLVESGGGLVKPGGSLKLSCAASGFTFSDYAMSWVRQTPEKRLEWVAYISSGGGNTYYLDSVKGRFTISRDNAKNTLYLQMSSLRSEDTALFYCAREESYNYYRHFFDYWGQGTTLTVSS

SEQ ID NO: 22 - protein, 110 aa, synthetic construct
EIVLTQSPALMAASPGEKVTITCSVSSSISSSSLHWYQQKSGASPKPWIYGTSNLASGVPVRFSGSGSGTSFSLTISSMEAEDAATYYCQQWSSYPPILTFGAGTKLELK

SEQ ID NO: 23 - protein, 116 aa, synthetic construct
QGQLQQSGAELMKPGASVKISCRGTGYTFRSYWIEWVKQRPGHGLEWIGEILPGIGSTYYNEKFKGKATFTADTSSNTAYMQLSSLTSDDSAVYFCARGGRDFDYWGQGTTLTVSS

SEQ ID NO: 24 - protein, 107 aa, synthetic construct
DIQMTQTTSSLSASLGDRVTISCRASQDINNYLNWYQQKPDGTVKLLIYDTSRLHSGVPSRFSGSGSGTDFSLTISNLEPEDIATYYCQQYSKLPYTFGSGTKLEIK

SEQ ID NO: 25 - protein, 117 aa, synthetic construct
EVQLQQSGPELVKPGASVKMSCKASGFTFTNYVFHWVKQKPGQGLEWIGYINPYNDGTKYNERFKGKATLTSDKSSSTAYMELSSLTSEDSAVYFCARVGNYVFAYWGQGTLVTVSA

SEQ ID NO: 26 - protein, 111 aa, synthetic construct
DIVLTQSPASLAVSLGQSVTISCRASESVEYYGTSLMQWYQQKPGQPPKLLIYGASNVESGVPARFSGSGSGTDFSLNIHPVEEDDIAMYFCQQSRKVPWTFGGGTKLEIK

SEQ ID NO: 27 - protein, 119 aa, synthetic construct
EVQLQQSGPELVKPGASVKISCKASGYSFTGYFLNWVKLSHGKSLEWIGRINPYNGDTFYNQKFKGKATLTVDKSSSTAHMEVRSLTSEDSAVYYCARREGNLYSMDYWGQGTSVTVSS

SEQ ID NO: 28 - protein, 113 aa, synthetic construct
DIVMTQSPSSLTVTAGEKVTLSCKSSQSLLNSGNQKNFLTWYQQKPGQPPKLLIYWASTRESGVPDRFTGSGSGTDFTLAISSVQAEDLAVYYCQNDYSYPLTFGAGTKLELK

SEQ ID NO: 29 - protein, 121 aa, synthetic construct
QVQLVQSGAEVKKPGSSVKVSCKASGYSFTGYYMNWVRQAPGQGLEWMGEINPSIDVATYNQKFKARVTITVDKSTSTAYMELSSLRSEDTAVYYCASSYLLWSSGYFDVWGQGTTVTVSS

SEQ ID NO: 30 - protein, 112 aa, synthetic construct
DVVMTQSPLSLPVTLGQPASISCTSSQSLLYTNGKTYLNWLQQRPGQSPRRLISLVSKLDSGVPDRFSGSGSGTDFTLKISRVEAEDVGVYYCFQSTHFPFTFGQGTKLEIK

SEQ ID NO: 31 - protein, 121 aa, synthetic construct
QVQLVQSGAEVKKPGSSVKVSCKASGYSFTGYYMNWVRQAPGQGLEWMGEINPSIDVATYNQKFKARATITVDKSTSTAYMELSSLRSEDTAVYYCASSYLLWSSGYFDVWGQGTTVTVSS

SEQ ID NO: 32 - protein, 112 aa, synthetic construct; identical to SEQ ID NO: 30

SEQ ID NO: 33 - protein, 121 aa, synthetic construct
QVQLVQSGAEVKKPGSSVKVSCKASGYSFTGYYMNWVRQAPGQSLEWMGEINPSIDVATYNQKFKARVTITVDKSTSTAYMELSSLRSEDTAVYYCASSYLLWSSGYFDVWGQGTTVTVSS

SEQ ID NO: 34 - protein, 112 aa, synthetic construct; identical to SEQ ID NO: 30

SEQ ID NO: 35 - protein, 129 aa, synthetic construct
QVQLQESGPGLVKPPGTLSLTCAVSGGSISSSNWWSWVRQPPGKGLEWIGEIYHGGYTNYNPSLKSRVTISIDKSKNQFSLKLSSVTAADTAVYYCTRDMGANYYGSGNYYDVGWFDPWGQGTLVTVSS

SEQ ID NO: 36 - protein, 107 aa, synthetic construct; identical to SEQ ID NO: 2

SEQ ID NO: 37 - protein, 129 aa, synthetic construct
QVQLQESGPGLVKPPGTLSLTCAVSGGSISSSNWWSWVRQPPGKGLEWIGEIYHAGYTNYNPSLKSRVTISIDKSKNQFSLKLSSVTAADTAVYYCTRDMGINYYGSGNYYDVGWFDPWGQGTLVTVSS

SEQ ID NO: 38 - protein, 107 aa, synthetic construct; identical to SEQ ID NO: 2

SEQ ID NO: 39 - protein, 129 aa, synthetic construct
QVQLQESGPGLVKPPGTLSLTCAVSGGSISSSNWWSWVRQPPGKGLEWIGEIYHMGYTNYNPSLKSRVTISIDKSKNQFSLKLSSVTAADTAVYYCTRDMGANYYGSGNYYDVGWFDPWGQGTLVTVSS

SEQ ID NO: 40 - protein, 107 aa, synthetic construct; identical to SEQ ID NO: 2

SEQ ID NO: 41 - protein, 119 aa, synthetic construct
EVQLVESGGGLVQPGGSLRLSCAASGYTFTNFGMNWVRQAPGKGLEWVAWINTNTGEPRYAEEFKGRFTISRDNAKNSLYLQMNSLRAEDTAVYYCARDWDGAYFFDYWGQGTLVTVSS

SEQ ID NO: 42 - protein, 107 aa, synthetic construct
DIQMTQSPSSLSASVGDRVTITCKASQSVSNDVAWYQQKPGKAPKLLIYFATNRYTGVPSRFSGSGYGTDFTLTISSLQPEDFATYYCQQDYSSPWTFGQGTKVEIK

SEQ ID NO: 43 - protein, 122 aa, synthetic construct
EVQLEESGGGLVKPGGSLRLSCAASGIDLSHYVVGWVRQAPGKGLEWVSIIYGSGRTYYANWAKGRFTISRDNSKNTLYLQMNSLRAEDTAVYYCARDASVSVYYWGYFDLWGRGTLVTVSS

SEQ ID NO: 44 - protein, 110 aa, synthetic construct
DIQMTQSPSTLSASVGDRVTITCQASQSIGSELAWYQQKPGKAPKLLIYRASTLESGVPSRFSGSGSGTEFTLTISSLQPDDFATYYCQQGYTYSEIDNAFGQGTKVEIK

SEQ ID NO: 45 - protein, 121 aa, synthetic construct
EVQLVESGGDLAQPGGSLRLSCAVSGIDLSSYGMGWVRQAPGKGLEWVSIISRNSVTYYATWAKGRFTISRDNSKNTVYLQMTSLRAEDTALYFCARRATYSGALGYFDIWGQGTLVTVSS

SEQ ID NO: 46 - protein, 110 aa, synthetic construct
EIVMTQSPSSLSASVGDRVTITCQASENIYSTLAWYQQKPGKAPKLLIYDAFDLASGVPSRFSGSGSGTDYTLTISSLQPEDFATYYCQQGYSGTNVDNAFGQGTKLEIK

SEQ ID NO: 47 - protein, 9 aa, synthetic construct
GGSISSSNW

SEQ ID NO: 48 - protein, 6 aa, synthetic construct
SSNWWS

SEQ ID NO: 49 - protein, 8 aa, synthetic construct
GGSISSSN

SEQ ID NO: 50 - protein, 11 aa, synthetic construct
GGSISSSNWWS

SEQ ID NO: 51 - protein, 7 aa, synthetic construct
SSSNWWS

SEQ ID NO: 52 - protein, 7 aa, synthetic construct
IYHGGYT

SEQ ID NO: 53 - protein, 16 aa, synthetic construct
EIYHGGYTNYNPSLKS

SEQ ID NO: 54 - protein, 5 aa, synthetic construct
YHGGY

SEQ ID NO: 55 - protein, 9 aa, synthetic construct
EIYHGGYTN

SEQ ID NO: 56 - protein, 12 aa, synthetic construct
WIGEIYHGGYTN

SEQ ID NO: 57 - protein, 22 aa, synthetic construct
TRDMGANYFGSGNYYDVGWFDP

SEQ ID NO: 58 - protein, 20 aa, synthetic construct
DMGANYFGSGNYYDVGWFDP

SEQ ID NO: 59 - protein, 21 aa, synthetic construct
TRDMGANYFGSGNYYDVGWFD

SEQ ID NO: 60 - protein, 6 aa, synthetic construct
QGIRSY

SEQ ID NO: 61 - protein, 11 aa, synthetic construct
RASQGIRSYLA

SEQ ID NO: 62 - protein, 7 aa, synthetic construct
RSYLAWY

SEQ ID NO: 63 - skipped

SEQ ID NO: 64 - protein, 7 aa, synthetic construct
EASTLQS

SEQ ID NO: 65 - protein, 10 aa, synthetic construct
VLIYEASTLQ

SEQ ID NO: 66 - protein, 9 aa, synthetic construct
QQLNNYPRT

SEQ ID NO: 67 - protein, 8 aa, synthetic construct
QQLNNYPR

SEQ ID NO: 68 - protein, 8 aa, synthetic construct
GYSFTGYY

SEQ ID NO: 69 - protein, 5 aa, synthetic construct
GYYMN

SEQ ID NO: 70 - protein, 7 aa, synthetic construct
GYSFTGY

SEQ ID NO: 71 - protein, 10 aa, synthetic construct
GYSFTGYYMN

SEQ ID NO: 72 - protein, 6 aa, synthetic construct
TGYYMN

SEQ ID NO: 73 - protein, 10 aa, synthetic construct
GYSFTGYYMN

SEQ ID NO: 74 - protein, 8 aa, synthetic construct
INPSIDVA

SEQ ID NO: 75 - protein, 17 aa, synthetic construct
EINPSIDVATYNQKFKA

SEQ ID NO: 76 - protein, 6 aa, synthetic construct
NPSIDV

SEQ ID NO: 77 - protein, 10 aa, synthetic construct
EINPSIDVAT

SEQ ID NO: 78 - protein, 13 aa, synthetic construct
WIGEINPSIDVAT

SEQ ID NO: 79 - protein, 14 aa, synthetic construct
ASSYLLWSSGYFDV

SEQ ID NO: 80 - protein, 12 aa, synthetic construct
SYLLWSSGYFDV

SEQ ID NO: 81 - protein, 13 aa, synthetic construct
ASSYLLWSSGYFD

SEQ ID NO: 82 - protein, 11 aa, synthetic construct
QSLLYTNGKTY

SEQ ID NO: 83 - protein, 16 aa, synthetic construct
TSSQSLLYTNGKTYLN

SEQ ID NO: 84 - protein, 12 aa, synthetic construct
LYTNGKTYLNWL

SEQ ID NO: 85 - skipped

SEQ ID NO: 86 - protein, 7 aa, synthetic construct
LVSKLDS

SEQ ID NO: 87 - protein, 10 aa, synthetic construct
RLISLVSKLD

SEQ ID NO: 88 - protein, 9 aa, synthetic construct
FQSTHFPFT

SEQ ID NO: 89 - protein, 8 aa, synthetic construct
FQSTHFPF

SEQ ID NO: 90 - protein, 7 aa, synthetic construct
IYHAGYT

SEQ ID NO: 91 - protein, 7 aa, synthetic construct
IYHMGYT

SEQ ID NO: 92 - protein, 7 aa, synthetic construct
IYHRGYT

SEQ ID NO: 93 - protein, 22 aa, synthetic construct
TRDMGINYFGSGNYYDVGWFDP

SEQ ID NO: 94 - protein, 22 aa, synthetic construct
TRDMGVNYFGSGNYYDVGWFDP

SEQ ID NO: 95 - protein, 22 aa, synthetic construct
TRDMGANYYGSGNYYDVGWFDP

SEQ ID NO: 96 - protein, 22 aa, synthetic construct
TRDMGINYWGSGNYYDVGWFDP

SEQ ID NO: 97 - protein, 22 aa, synthetic construct
TRDMGINYYGSGNYYDVGWFDP

SEQ ID NO: 98 - protein, 22 aa, synthetic construct
TRDMGVNYYGSGNYYDVGWFDP

SEQ ID NO: 99 - protein, 6 aa, synthetic construct
WGIRSY

SEQ ID NO: 100 - protein, 6 aa, synthetic construct
YGIRSY

SEQ ID NO: 101 - protein, 330 aa, Homo sapiens [sequence omitted]

SEQ ID NO: 102 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 107 aa, Homo sapiens [sequence omitted]

SEQ ID NO: 104 - protein, 420 aa, Homo sapiens
MPGGCSRGPAAGDGRLRLARLALVLLGWVSSSSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 105 - protein, 325 aa, Homo sapiens
SSSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTS

SEQ ID NO: 106 - protein, 420 aa, synthetic construct
MPGGCSRGPAAGDGRLRLARLALVLLGWVSSSSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALRSGLALRGLTRLELASNHFLFLPRDLLAQLPSLRYLDLRNNSLVSLTYASFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTSYVFLGIVLALIGAIFLLVLYLNRKGIKKWMHNIRDACRDHMEGYHYRYEINADPRLTNLSSNSDV

SEQ ID NO: 107 - protein, 426 aa, synthetic construct
MPGAGSRGPSAGDGRLRLARLALVLLGWVSASAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLEELILNHIVPPEDQRQNGSFEGMVAFEGMVAAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 108 - protein, 233 aa, synthetic construct
QVQLVQSGAEVKKPGSSVKVSCKASGYSFTGYYMNWVRQAPGQGLEWMGEINPSIDVATYNQKFKARVTITADKSTSTAYMELSSLRSEDTAVYYCARSYLLWSSGYFDVWGQGTTVTVSSDVVMTQSPLSLPVTLGQPASISCTSSQSLLYTNGKTYLNWFQQRPGQSPRRLIYLVSKLDSGVPDRFSGSGSGTDFTLKISRVEAEDVGVYYCFQSTHFPFTFGQGTKLEIK

SEQ ID NO: 109 - protein, 112 aa, synthetic construct
DVVMTQSPLSLPVTLGQPASISCTSSQSLLYTNGKTYLNWFQQRPGQSPRRLIYLVSKLDSGVPDRFSGSGSGTDFTLKISRVEAEDVGVYYCFQSTHFPFTFGQGTKLEIK